(19) United States
(12) Patent Application Publication
(10) Pub. No.: US 2025/0034264 A1
(43) Pub. Date: Jan. 30, 2025

(54) REGULATING T CELL IMMUNE RESPONSE THROUGH IGF-I RECEPTOR (IGF-1R)

(71) Applicant: The Regents of the University of Michigan, Ann Arbor, MI (US)

(72) Inventor: Terry J. Smith, Ann Arbor, MI (US)

(21) Appl. No.: 18/559,732

(22) PCT Filed: May 11, 2022
(86) PCT No.: PCT/US2022/028784

§ 371 Date: Nov. 8, 2023

Related U.S. Application Data
(60) Provisional application No. 63/187,295, filed on May 11, 2021.

(51) Int. Cl. C07K 16/28; A61K 31/4985; A61K 39/00

(52) U.S. Cl. CPC C07K 16/2863; A61K 31/4985; A61K 2039/505

(57) ABSTRACT

The present disclosure provides methods for treating or preventing diseases or disorders (e.g., inflammatory, fibrosis, and autoimmune diseases or disorders) with IGF-1R inhibitors. The diseases or disorder may be selected from: neuromyelitis optica spectrum disorder (NMOSD), myasthenia gravis, pemphigus, rheumatoid arthritis, fibrosing or sclerosing cholangitis, pulmonary fibrosis, Peyronie's disease, Dupuytren's contracture, carpal tunnel syndrome, dermatomyositis, lethal midline granuloma syndrome (LMG), (Type I diabetes, ankylosing spondylitis, polymyalgia rheumatica, systemic lupus erythematosus (SLE), and immune thrombocytopema (ITP) psoriasis, scleroderma, autoimmune hemolytic anemia, or a combination thereof.

### CROSS-REFERENCE TO RELATED APPLICATIONS

This application claims the benefit of U.S. Provisional Application No. 63/187,295, filed May 11, 2021, the content of which is herein incorporated by reference in its entirety.

### STATEMENT REGARDING FEDERALLY-SPONSORED RESEARCH

This invention was made with Government support under contract EY08976 awarded by the National Institutes of Health. The Government has certain rights in the invention.

### FIELD

The present invention relates to methods of treating diseases and disorders characterized by abnormal differentiation and expansion of antigen specific T helper cells (e.g., Th1 and Th17 cells) with IGF-1R inhibitors.

### SEQUENCE LISTING STATEMENT

The text of the computer readable sequence listing filed herewith, titled “39476-601_SEQUENCE-LISTING_ST25”, created May 11, 2022, having a file size of 3,182 bytes, is hereby incorporated by reference in its entirety.

### BACKGROUND

The insulin-like growth factor-1 receptor (IGF-1R) has been implicated broadly in the regulation of both normal immunity and autoimmune diseases. Like many other membrane-spanning tyrosine kinase receptors, IGF-1R can form signaling complexes, either with other tyrosine kinase receptors or with G-protein coupled receptors. Several inhibitors of IGF-1R were developed as treatment candidates for neoplastic diseases more than a decade ago; however, results of multiple early stage clinical trials proved disappointing. Among these agents, teprotumumab, a β-arrestin biased IGF-1R agonist, was very recently approved by the U.S. Food and Drug Administration for the treatment of thyroid-associated ophthalmopathy (TAO) based on results from two double-masked, placebo-controlled randomized clinical trials.

### SUMMARY

Provided herein are methods for treating or preventing a disease or disorder in a subject comprising administering to the subject a therapeutically or prophylactically effective amount of at least one insulin-like growth factor-1 receptor (IGF-1R) inhibitor or a composition thereof, wherein the disease or disorder does not comprise Graves' disease. In some embodiments, the disease or disorder is characterized by antigen presenting cells (e.g., fibrocytes and dendritic cells) driving abnormal differentiation and expansion of antigen specific T helper cells (e.g., Th1 and Th17 cells). In some embodiments, administration of the at least one IGF-1R inhibitor attenuates antigen presentation, decreases tissue remodeling, or a combination thereof. In some embodiments, the IGF-1R inhibitor further reduces the levels of interferon 7 and IL-17A, thereby attenuating drivers of tissue remodeling and inflammatory reactivity.

In some embodiments, the IGF-1R inhibitor attenuates basal and TSH-inducible autoimmune regulator protein (AIRE), thyroglobulin (Tg), sodium iodide symporter (NIS), thyroperoxidase (TPO), IL-10 and B-cell activating factor (BAFF) levels. In some embodiments, the IGF-1R inhibitor downregulates IL-23p19 expression/induction while enhancing IL-12p35, intracellular and secreted IL-1 receptor antagonists and Slit2. In some embodiments, the IGF-1R inhibitor increases HAS2 expression and hyaluronan (HA) production.

In some embodiments, the disease or disorder comprises an inflammatory, fibrosis, and/or autoimmune disease. In some embodiments, the disease or disorder is selected from the group consisting of: neuromyelitis optica spectrum disorder (NMOSD), myasthenia gravis, pemphigus, rheumatoid arthritis, fibrosing or sclerosing cholangitis, pulmonary fibrosis, Peyronie's disease, Dupuytren's contracture, carpal tunnel syndrome, dermatomyositis, lethal midline granuloma syndrome (LMG), Type I diabetes, ankylosing spondylitis, polymyalgia rheumatica, systemic lupus erythematosus (SLE), immune thrombocytopenia (ITP) psoriasis, scleroderma, autoimmune hemolytic anemia, and combinations thereof. In some embodiments, IGF-1R inhibition reduces the untoward consequences in infection-driven tissue activation and organ system failure.

In some embodiments, the IGF-1R inhibitor comprises an antibody against IGF-1R or a small molecule IGF-1R tyrosine kinase inhibitor. In some embodiments, the IGF-1R inhibitor comprises teprotumumab, linsitinib, IGF-1R Antibody 1H7, or a combination thereof.

In some embodiments, the methods further comprise administration of at least one additional therapeutic agent. In some embodiments, the at least one additional therapeutic agent comprises a corticosteroid, an immunosuppressant, rituximab, an anti-inflammatory agent, an antibiotic, an opioid antagonist, a vitamin or nutritional supplement, or any combination thereof.

Other aspects and embodiments of the disclosure will be apparent in light of the following detailed description and accompanying figures.

### BRIEF DESCRIPTION OF THE DRAWINGS

FIGS. 1A-1D show divergent expression patterns in vitro of MHC 11, CD80, CD86, and PD-L1 in CD34-fibrocytes and GD-OF. Isolated peripheral blood mononuclear cells (PBMCs) were incubated and substratum-adherent CD34⁺ fibrocytes were analyzed for MHC-II, CD80, CD86, and PD-L1 mRNA and cell-surface levels in fibrocytes and GD-OF (FIG. 1A). RNA was extracted from confluent fibrocyte and GD-OF monolayers and reverse transcribed cDNAs were subjected to RT-qPCR. Signals were normalized to respective GAPDH (mean±SD, n=3 independent determinations). Cell-surface displayed proteins were assessed by flow cytometry using the gating strategies shown in FIG. 8A. Dotted histograms represent isotype control Ab staining. FIG. 1B is a schematic of parental GD-OF (admixture of CD34⁺ OF and CD34⁻ OF) staining with anti-CD34 mAb or isotype control mAb for 30 m. Cells were subjected to cytometry cell sorting into pure CD34⁺ OF, CD34⁻ OF or mock sorted into parental GD-OF. These were incubated for 48 h. FIG. 1C is a graph of cellular mRNA from monolayers depicted in FIG. 1B subjected to RT-PCR for the respective gene targets. Data are expressed as the mean f SD of three independent determinations. FIG. 1D are graphs of parental, pure CD34⁺ OF and CD34⁻ OF subsets analyzed by flow cytometry for cell-surface MHC II, CD80, CD86, and PD-L1 display. Data represent mean fluorescence intensity compared with that in isotype controls in the CD34/Col1/CXCR4 gate. Experiments were performed three times. ** p<0.01; *** p<0.001, and **** p<0.0001.

FIGS. 2A and 2B show that Slit2 attenuates MHC II, CD80, CD86, and PD-L1 expression in fibrocytes while knocking down Slit2 expression in GD-OF enhances expression of each protein. Cultured fibrocytes were treated with rhSlit2 (50 ng/ml) for 5 to 7d, cellular RNA was extracted and reverse transcribed, the resultant cDNAs were subjected to RT-qPCR for the respective targets and normalized to their respective GAPDH signals (FIG. 2A). Sister cultures were stained with anti-human MHC II, CD80, CD86, and PD-L1 mAbs or control isotype mAbs and underwent flow cytometric analyses. Parental GD-OF were transfected with either siRNA targeting Slit2 or scrambled siRNA control (each 3 μg/ml) for 4 to 5 d, cellular RNA was subjected to RT-PCR for the targets indicated and normalized to their respective GAPDH signals (FIG. 2B). Sister cultures were stained with the mAbs used in FIG. 2A and underwent flow cytometric analysis for the respective proteins in the CD34/Col1/CXCR4 gate. Data are from three independent determinations and reflect results from three experiments performed. * p<0.05; ** p<0.01 and *** p<0.001.

FIGS. 3A-3F show IGF-1R inhibition and knock-down resulted in reduced levels of MHC II, CD80, CD86, and PD-L1. IL-17A and INFγ levels in fibrocyte-adherent CD4⁺ T cells are also reduced. Time-dependent effects in vitro of teprotumumab (Tepro, 50 μg/mL), 1H7 (5 μg/mL), human Isotype IgG1 (5 μg/mL), and linsitinib (1 μM) on levels of mRNA encoding MHC II, CD80, CD86 and PD-L1 (FIG. 3A) or on levels of respective cell-surface displayed proteins (FIG. 3B). Flow analysis results denote mean fluorescence intensity compared with isotype controls in the CD34⁺/CXCR4⁺/Col⁺ gate. PBMCs were incubated in the absence or presence of above agents for the graded intervals indicated along the abscissas. All cultures were harvested after a 9-day incubation, when cellular RNA was extracted, reversed transcribed and subjected to RT-qPCR while sister culture monolayers were stained with mAbs for each target or an isotype control mAb. Fibrocyte cultures were transfected with IGF-1R-targeting siRNA or scrambled siRNA controls (each 3 μg/ml), incubated for 5-7 d (FIG. 3C). Monolayers were treated with either teprotumumab, its isotype IgG, or ML224 for 9 d. Cellular RNA was reverse transcribed and subjected to RT-PCR and target levels normalized to respective GAPDH signals (FIG. 3D). IL-17A and INFγ released into the medium from fibrocyte/adherent T cell monolayers were analyzed by cytokine-specific ELTSAs and normalized to respective cell layer protein content (FIG. 3E). Fibrocyte/adherent T cell cultures (Upper panel) and purified T cells (lower panel) were treated with Tepro (50 μg/mL) or human Isotype IgG1 for 9 d and subjected to flow analysis for IL-17A and INFγ. Results were denoted mean fluorescence intensity compared with isotype controls in the CD3⁺/CD4⁺ gate for IL-17A and INFγ (FIG. 3F). Results are from one experiment, representative of three performed. PCR results are expressed as the mean±SD of three independent determinations from one representative study of three performed. * p<0.05; ** p<0.01; and *** p<0.001

FIGS. 4A-4F show the effects of teprotumumab (Tepro), 1H7, and linsitinib on basal and TSH-dependent MHC II, CD80, CD86, PD-L1 expression in fibrocytes. Time-dependent effects in vitro of TSH 5 mIU/mL) on steady-state mRNA (FIG. 4A) and cell-surface protein display (FIG. 4B), and effects of M22 (1 μg/mL, FIG. 4C) and IGF-1 (10 nM, FIG. 4D) on surface-displayed proteins. FIG. 4E shows the impact of Tepro (50 μg/mL), isotype IgG control mAb, Lin (1 μM) and 1H7 (5 μg/mL) on the mRNA inductions by bTSH or M22 for the treatment times indicated along the abscissas. FIG. 4F is flow cytometric analysis of respective proteins levels in untreated fibrocytes and those treated with bTSH, Tepro or the two compounds in combination. Monolayers were collected, cellular RNA extracted, reverse transcribed and cDNAs subjected to RT-PCR analysis. Sister culture monolayers were collected, cells were stained with mAbs for each target or an isotype control mAb and subjected to flow cytometric analysis. Flow analysis results denote mean fluorescence intensity compared with isotype controls in the CD34⁺/CXCR4⁺/Col⁺ gate. A total of three experiments were performed. * p<0.05; ** p<0.01; *** p<0.001 and **** p<0.0001

FIGS. 5A-5C show the effects of teprotumumab on basal and bTSH-induced gene promoter activities in GD-OF compared to fibrocytes, gene transcription and mRNA stabilities of MHC II, CD80, CD86, PD-L1 in fibrocytes. Confluent GD-OF and fibrocyte monolayers were treated with human Isotype IgG (5 μg/ml) or teprotumumab (50 μg/mL) for 3 d and transfected with a target gene promoter fragment (MHC-2, CD80, CD86, PD-L1, or negative control clone PG04). Following a 3-4 d incubation, they were treated without or with bTSH (5 mU/mL) for 2 h. Monolayers were rinsed, processed, and analyzed using the Secrete-Pair Dual Luminescence Assay Kit for promoter activity according to manufacturer's instructions (FIG. 5A). Values were normalized to internal controls. Fibrocyte monolayers were cross-linked with 16% formaldehyde isolated chromatin fragment and immunoprecipitated with anti-human HLA-DPB1, anti-CD80, anti-CD86, or anti-PD-L1 using the Pierce™ Agarose Chip Kit. Isolated DNA was subjected to RT-PCR using EpiTect ChIP qPCR Primer Assay (Qiagen) for each target (FIG. 5B). Each datum point represents values from three independent determinations from one of three experiments performed. *, p<0.05; ** p<0.01; and ***p<0.001. Fibrocyte cultures were pretreated with bTSH for 6 h. At time “0” all culture wells received DRB (20 μg/mL) without or with the agents indicated, including teprotumumab (50 μg/ml) or human Isotype IgG (5 μg/ml) alone or in combination with bTSH (5 mIU/mL) for the intervals indicated along the abscissas. RNA was extracted, reverse transcribed, and subjected to real-time quantitative PCR for each target (FIG. 5C). Values were generated according to the protocol provided by the supplier and normalized to respective GAPDH signals. Measures of MCH II, CD80, CD86, and PD-L1 were summarized between control, TSH, and Tepro treatment groups at each time point using mean, standard deviations (SD), and medians. Measures were compared between groups, stratified by time point, using ANOVA. Significant ANOVA results were followed with post-hoc pairwise comparisons using 2-sample t-tests. Linear regression was used to model slopes of MCH U, CD80, CD86, and PD-L1 measures over time and assess differences between groups (control, TSH, Tepro). A regression spline was used to allow for different slopes during the time immediately following treatment (time 0-1 hour) versus extended time on treatment (time 1-12 hours), as the initial effect of treatment was substantial. Slopes were tested for differences between treatment groups and estimates are reported with 95% confidence intervals (CI). SAS version 9.4 (SAS Institute, Cary, NC) was used for statistical analysis. Each datum point represents values from three independent determinations from one of three experiments performed. P<0.04 for all datum points except 12 h point for CD86 (p<0.08) by Kruskal-Wallis.

FIGS. 6A-6C show a comparison of teprotumumab effects in cultured fibrocytes/adherent T cells on MHC II, CD80, CD86, and PD-L1 expression in CD34⁺CXCR4⁺ fibrocytes and interferon γ and IL-17A in CD3⁺CD4⁺ T cells from healthy individuals and those with Graves' disease. Cultured peripheral blood mononuclear cells (PBMCs) from healthy control donors (n=31) and patients with Graves' disease (n=28) were treated with human Isotype IgG (5 μg/ml) or teprotumumab (50 μg/mL) for 9 d. Monolayers were stained with anti-human MHC II, CD80, CD86, PD-L1, CD34, CXCR4, CD3 and CD4 mAbs for 30 min as described in Methods. Cells were analyzed by flow cytometry for the cell markers indicated. Data represent mean fluorescence intensity compared with isotype controls in CD34⁺/CXCR4⁺ gate (FIG. 6A) and CD3⁺/CD4⁺ gate (FIGS. 6B and 6C). Conditioned culture media at day 9 were collected and analyzed for IL-17A and INFγ using cytokine-specific ELISAs (FIG. 6D). Values were normalized to respective cell layer protein content. A total of 59 independent studies, each involving a different individual, were performed and included in the data set shown. * p<0.05; ** p<0.01; *** p<0.001 and **** p<0.0001 by paired t-test followed by Tukey's multi-comparison test.

FIGS. 7A-7B show baseline and teprotumumab post-treatment MHC H, CD80, CD86, PD-L1 levels in circulating CD34⁺CXCR4⁺ fibrocytes and interferon γ and IL-17A protein expression in circulating CD3⁺CD4⁺ T cells. Cryopreserved peripheral blood mononuclear cells (PBMCs) collected at study baseline (pretreatment) and after a 24-week treatment with either placebo (n=33) or teprotumumab (n=32) administered as IV infusions every three weeks in patients with moderate to severe TAO in a therapeutic phase 2 trial (20). PBMCs were thawed, rinsed, and stained with the following mAbs: anti-human CD34, CXCR4, CD3, CD4, MHC II, CD80, CD86, PD-L1, interferon γ (INFγ) or IL-17A, as described in Methods. Stained cells were rinsed and subjected to flow cytometric analysis. The gating strategies used for these analyses are shown in FIG. 8. Mean fluorescence intensity (MFI) was compared with isotype controls and analyzed in the CD34/CXCR4 (FIG. 7A) or CD3/CD4 (FIG. 7B) gate. * p<0.05; ** p<0.01; ***p<0.001 and **** p<0.0001 by paired t-test followed by Tukey's multi-comparison test.

FIGS. 8A-8D show gating strategy for fibrocytes (FIG. 8A, upper panel), T cells (FIG. 8A, lower panel) and GD-OF flow cytometric analysis (FIG. 8A). Fibrocyte gating was fixed to the CD45⁺/CD34⁺/Col1⁺/CXCR4⁺ cell populations while T cell gating was fixed to CD3⁺/CD4⁺ double-stain analysis. GD-OF gating fixed to CD34⁺ subpopulation. All gating was fixed according to respective isotype mAb controls. FIGS. 8C and 8D shows positive controls for siRNA knockdown of Slit2 and IGF-1R targets, respectively. siRNA knockdown of target genes was compared with those from a scramble control (siRNA) for 4 d. RNA was reverse transcribed and resultant cDNA subjected to RT-qPCR and normalized to the respective cellular GAPDH (mean±SD, n=3 independent determinations). ***p<0.001

FIGS. 9A-9D are graphs showing the effect of teprotumumab on bovine thyrotropin or thyroid-stimulating hormone (bTSH) induced thyroid specific genes thyroglobulin (Tg), sodium iodide symporter (NIS), thyroperoxidase (TPO), and autoimmune regulator protein (AIRE) in fibrocytes. Confluent fibrocyte cultures were treated with or without teprotumumab (50 μg/ml) and human Isotype IgG for 7-9 d, followed by bTSH (5 mIU/mL) for 6 h. Monolayers were washed with PBS and RNA was extracted and reverse transcribed. Harvested cDNA was subjected to quantitative real-time PCR for AIRE (FIG. 9A), Tg (FIG. 9B), TPO (FIG. 9C), and NIS (FIG. 9D) mRNA levels. Values were normalized to their respective GAPDH levels and are expressed as the mean f SD of three independent determinations. Experiments were repeated three times. *, P<0.05; **, p<0.01.

FIGS. 10A-10F are graphs showing the divergent effect of teprotumumab on bTSH induced IL-23p19 and IL-12p35 expression on fibrocytes and GD-OF. Rinsed fibrocyte and GD-OF monolayers were incubated with or without 50 μg/ml teprotumumab (FIGS. 10A and 10B) or 1 μM linsitinib (FIGS. 10C and 10D) and human Isotype IgG for 7-9 d, and bTSH (5 mIU/mL) was added for 6 h. Harvested RNA was reverse transcribed and isolated cDNA was subjected to quantitative real-time PCR for IL-23p19 and IL-12p35 mRNA levels. Values were normalized to their respective GAPDH levels and are expressed as the mean±SD of three independent determinations. Experiments were repeated three times. FIGS. 10E and 10F are graphs showing the levels of IL-23 protein when rinsed fibrocyte and GD-OF monolayers were incubated with or without 50 μg/ml teprotumumab. **, p<0.01; ***. P<0.001.

FIGS. 11A-11F are graphs showing teprotumumab attenuates bTSH induced IL-10 while enhancing icIL-1RA and sIL-1RA expression in fibrocytes and GD-OF. Fibrocyte cultures were treated with teprotumumab (50 μg/ml), rhSlit2 (50 ng/ml) or human Isotype IgG for 7-9 d, with bTSH (5 mIU/mL) for final 6 h. RNA was extracted and reverse transcribed. Isolated cDNA was subjected to quantitative real-time PCR for IL-10 (FIGS. 11A and 11B), icIL-1RA (FIGS. 11C and 11D). Media and cellular proteins were collected and subjected to specific ELISAs for icIL-1RA and sIL-1RA (FIGS. 11E, 11F, 11G, and 11H). PCR values were normalized to their respective GAPDH levels. icIL-1RA and sIL-1RA protein levels were normalized to cellular protein levels. Data are expressed as the mean±SD of three independent determinations. Experiments were repeated three times. **, p<0.01; ***. P<0.001.

FIGS. 12A-12F are graphs showing the effect of bTSH, teprotumumab, and Slit2 on B-cell activating factor (BAFF) expression in fibrocytes and GD-OF. In FIGS. 12A and 12B, cultures were treated for the graded intervals indicated along the abscissas with bTSH (5 mIU/mL). Cellular RNA was harvested and subjected to reverse transcription by RT-PCR (FIG. 12A). Values were normalized to their respective GAPDH levels Medium was assayed for BAFF levels with an ELISA (FIG. 12B). Values were normalized to respective monolayer protein content. Data are expressed as mean f SD of triplicate determinations; *, p<0.05; **, p<0.01; ***, p<0.001. In FIGS. 12C-12F, bTSH, teprotumumab (50 μg/ml) or recombinant human Slit2 (rhSlit2) (50 ng/ml) were added to culture media for the times indicated and analyzed for BAFF mRNA levels as in FIG. 12A. In FIGS. 12F, parental GD-OF strains were sorted into pure CD34⁺ and CD34⁻ subsets. Subsets were then cultured for 48 h, the final 6 h in the absence or presence of bTSH. Data were expressed as mean f SD of triplicate independent determinations from a single experiment representative of three conducted. *, p<0.05; **, p<0.01

FIGS. 13A-13B are graphs showing the effect of teprotumumab on bTSH induced hyaluronan (HA) production in GD-OF (FIG. 13A) and fibrocytes (FIG. 13B). GD-OF were treated with or without teprotumumab (50 μg/ml) or human Isotype IgG (5 μg/ml) for 7-9 d and stimulated with bTSH (5 mIU/mL) for graded intervals indicated along the abscissa for 0 to 96h. Media were collected and subjected to HA ELISA. Values were normalized to respective cell layer protein levels and expressed as the mean±SD of three independent determinations. Experiments were repeated three times. FIGS. 13C-13F are graphs showing the divergent pattern of hyaluronan synthase (HAS) isoenzyme induction by bTSH in fibrocytes versus GD-OF and effects of teprotumumab (FIGS. 13C and 13D) and linsitinib (FIGS. 13E and 13F). Confluent monolayers of GD-OF and fibrocytes remained untreated or received teprotumumab (50 μg/ml) or linsitinib (1 μM) and human Isotype IgG for 7-9 d. Some cultures were treated with bTSH (5 mIU/mL) for 6h. Cellular RNA was harvested mRNA was reverse transcribed, and cDNA subjected to quantitative real-time PCR for HAS1 and HAS2. Values were normalized to their respective GAPDH levels and are expressed as the mean±SD of three independent determinations. Experiments were repeated three times. *, P<0.05; **, p<0.01; ***. P<0.001.

FIGS. 14A-14B are graphs showing teprotumumab induces Slit2 and roundabout 1 (ROBO1) mRNA and Slit2 protein in GD-OF. Cultures were treated with either isotype control IgG or teprotumumab (50 μg/ml) for 3 d. Culture media were collected and subjected to Slit2-specific ELISA (FIG. 14A). Levels of Slit2 were normalized to respective cell layer protein content. Cellular RNA was harvested from rinsed cell layers and mRNA reverse transcribed, and cDNA subjected to quantitative real-time PCR for Slit2 and ROBO1 (FIG. 14B). Values were normalized to their respective GAPDH levels. * *, p<0.01, * **, p<0.001.

FIGS. 15A-15C are dose-response curves of teprotumumab effects on IL-6 expression/induction by bTSH (FIG. 15A); HAS2 expression/induction by bTSH (FIG. 15B); IL-12p35 expression/induction by bTSH (FIG. 15C). Following indicated treatments, cellular RNA was harvest from rinsed GD-OF cell layers, reversed transcribed and subjected to real-time PCR. Values were normalized to their respective GAPDH levels.

### DETAILED DESCRIPTION OF THE INVENTION

The present disclosure is directed to methods for treating or preventing inflammatory, fibrosis-causing and/or autoimmune disease with insulin-like growth factor-1 receptor (IGF-1R) inhibitors or by altering the gene expression/translation of IGF-1R or one or more of its downstream signaling partners. Teprotumumab and other specific IGF-1R inhibitors attenuate the production of proinflammatory cytokines by T cells through their actions on antigen presenting cells known as CD34+ fibrocytes. Teprotumumab and other specific IGF-1R inhibitors inhibit the expression and induction by bTSH of several thyroid-specific proteins, including TSHR, thyroglobulin (Tg), thyroperoxidase (TPO), sodium-iodide symporter (NIS) as well as autoimmune regulator protein (AIRE), HAS1, B cell activating factor (BAFF), soluble IL-1 receptor antagonist (sIL-1RA), IL-23 and IL-10 in fibrocytes and GD-OF (Graves' disease orbital fibroblasts). In contrast, the IGF-1R inhibitors enhanced IL-12p35 and intracellular IL-1RA (icIL-1RA) and HAS2 expression.

### 1. Definitions

To facilitate an understanding of the present technology, a number of terms and phrases are defined below. Additional definitions are set forth throughout the detailed description.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “treat,” “treating,” and the like means a slowing, stopping, or reversing of progression of a disease or disorder or reducing the severity or activity thereof when provided a compound or composition described herein to an appropriate control subject. The term also means a reversing of the progression of such a disease or disorder to a point of eliminating or greatly reducing the symptoms. As such, “treating” means an application or administration of the compositions described herein to a subject, where the subject has a disease or a symptom of a disease, where the purpose is to cure, heal, alleviate, relieve, alter, remedy, ameliorate, improve, or affect the disease or symptoms of the disease.

As used herein, the term “preventing” refers to partially or indefinitely delaying onset of a disease, disorder and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition.

A “subject” or “patient” may be human or non-human and may include, for example, animal strains or species used as “model systems” for research purposes, such a mouse model as described herein. Likewise, patient may include either adults, juveniles (e.g., children), or infants. Moreover, patient may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein, the terms “providing,” “administering,” and “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Type I Insulin-Like Growth Factor Receptor (IGF-1R) Inhibitors

Insulin-like growth factor 1 (IGF-1) and insulin-like growth factor 2 (IGF2) are small (˜7.5 kDa) ligands that play critical roles in many biological processes including proliferation and protection from apoptosis and normal somatic growth and development. IGFs are members of a ligand family that includes insulin. The functions of IGF-1, IGF-2, and insulin are mediated through association with the cell surface receptor tyrosine kinases (RTKs) insulin-like growth factor 1 receptor (IGF-1R) and insulin receptor (INSR). IGF-1R binds to IGF1 or IGF2 with high affinity, and binds to insulin at a low affinity. The binding between IGFs and IGF-1R increases the activity of receptor tyrosine kinases and induces auto-phosphorylation of the receptors or phosphorylation of internal related materials, thereby resulting in signal transduction.

IGF-1R inhibitors for targeting or inhibiting IGF-1R activity/expression are known in the art. In some embodiments, IGF-1R inhibitors block activation of or binding of an agonistic ligand to IGF-1R, inhibit the IGF-1R induced signaling cascade, or inhibit receptor tyrosine kinase activity. In some embodiments, IGF-1R inhibitors disrupt the expression of IGF-1R or one or more of downstream signaling partners of IGF-1R. Suitable IGF-1R inhibitors include, but are not limited to, gene silencing oligonucleotides (e.g., an siRNA, an antisense oligonucleotide, dominant-negative, a short-hairpin RNA, a miRNA, a dicer-substrate RNA, a DNAzyme, or an aptamer targeting the IGF-1R gene or the IGF-1R messenger RNA), an anti-IGF-1R antibody (e.g., a monoclonal, polyclonal, murine, chimeric, humanized, or human antibody targeting an IGF-1R epitope or IGF-1R ligand, thus interfering with IGF-1R activity or ligand binding), a small molecule inhibitor of IGF-1R (e.g., a tyrosine kinase inhibitor), an anti-IGF antibody, or another IGF-1R inhibiting agent (e.g., an IGF ligand trap, a dominant negative receptor).

In some embodiments, the IGF-1R inhibitor comprises an antibody against IGF-1R Anti-IGF-1R antibodies include, but are not limited to, teprotumumab, 1H7, ganitumab, AMG 479, figitumumab (CP-751,871), cixutumumab (IMC-A12), dalotuzumab (MK0646), RG1507/R1507, robatumumab (SCH 717454 or MK-7454), istiratumab (MM-141), AVE-1642, dusigitumab (MEDI-573), xentuzumab (BI 836845), BIIB022, and rhuMab IGFR

In some embodiments, the IGF-1R inhibitor comprises a small molecule inhibitor. In some embodiments, the IGF-1R inhibitor comprises an IGF-1R tyrosine kinase inhibitor. Suitable IGF-1R inhibitors include, but are not limited to, linsitinib (OSI-906), BMS-754807, INSM-18 (nordihydroguaiaretic acid (NDGA)), XL228, picropodophyllin (AXL1717), BMS-536924, NVP-ADW742, NVP-AEW541, GSK621659A, GSK1904529A, GSK1838705A, A-928605, TAE226, PQ 401, and AG1024 (tyrphostin).

### 3. Methods of Treating a Disease or Disorder

The disclosure provides methods of treating or preventing a disease or disorder in a subject comprising administering to the subject a therapeutically or prophylactically effective amount of at least one insulin-like growth factor-1 receptor (IGF-1R) inhibitor, as described above, or a composition thereof.

Compositions comprising an IGF-1R inhibitor may further comprise excipients or pharmaceutically acceptable carriers. The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, antibacterial and antifungal agents, isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, preservatives, and antioxidants. Techniques and formulations may be found, for example, in Remington's Pharmaceutical Sciences, 19th Edition (Mack Publishing Company, 1995). The route or administration and the form of the composition usually dictates the type of carrier to be used.

The compositions may be formulated for any appropriate manner of administration, and thus administered, including for example, oral, nasal, intraocular, intravenous, intravaginal, epicutaneous, sublingual, intracranial, intradermal, intraperitoneal, subcutaneous, intramuscular administration, or via inhalation. Techniques and formulations may generally be found in “Remington's Pharmaceutical Sciences,” (Meade Publishing Co., Easton, Pa.). Therapeutic or pharmaceutical compositions must typically be sterile and stable under the conditions of manufacture and storage.

The terms “effective amount” or “therapeutically effective amount,” as used herein, refer to a sufficient amount of the IGF-1R inhibitor or compositions thereof being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the IGF-1R inhibitor or compositions required to provide a decrease in disease symptoms.

The amount of the IGF-1R inhibitor or a composition thereof required for use in treatment or prevention will vary not only with the particular IGF-1R inhibitor or composition selected but also with the route of administration, the nature and/or symptoms of the disease and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, useful dosages of an IGF-1R inhibitor, or composition thereof, can be determined by comparing their in vitro activity, and in vivo activity in animal models.

Dosage amount and interval may be adjusted individually to provide plasma levels of the active agent which are sufficient to maintain the modulating effects, or minimal effective concentration (MEC). The MEC will vary for each IGF-1R inhibitor but can be estimated from in vivo and/or in vitro data. Dosages necessary to achieve the MEC will depend on individual characteristics and route of administration. However, bioassays can be used to determine plasma concentrations. Dosage intervals can also be determined using MEC value. IGF-1R inhibitors or a composition thereof should be administered using a regimen, which maintains plasma levels above the MEC for 10-90% of the time, preferably between 30-90% and most preferably between 50-90%. In cases of local administration or selective uptake, the effective local concentration of the IGF-1R inhibitor may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate, precluding toxicity. The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be also used in veterinary medicine for non-human subjects.

Insulin-like growth factor-1 receptor (IGF-1R) inhibitors or a composition thereof can be evaluated for efficacy and toxicity using known methods. For example, the toxicology of a particular IGF-1R inhibitor may be established by determining in vitro toxicity towards a cell line, such as a mammalian, and preferably human, cell line. The results of such studies are often predictive of toxicity in animals, such as mammals, or more specifically, humans. Alternatively, the toxicity of particular IGF-1R inhibitor in an animal model, such as mice, rats, rabbits, dogs, or monkeys, may be determined using known methods. The efficacy of a particular IGF-1R inhibitor may be established using several recognized methods, such as in vitro methods, animal models, or human clinical trials. When selecting a model to determine efficacy, the skilled artisan can be guided by the state of the art to choose an appropriate model, dose, route of administration and/or regime.

An effective amount of an insulin-like growth factor-1 receptor (IGF-1R) inhibitor, or a composition thereof, may be administered alone or in combination with at least one additional therapeutic agent. In some embodiments, the at least one additional therapeutic agent is administered prior to, concomitantly with, or following administration of the IGF-1R inhibitor. The additional therapeutic agent may include, for example, a corticosteroid, an immunosuppressant, rituximab, anti-inflammatory agent, an antibiotic, an opioid antagonist, a vitamin or nutritional supplement, or any combination thereof. The choice of additional therapeutic agents will not only depend on the particular disease or disorder but also the nature and/or symptoms of the disease and the age and condition of the patient.

The disease or disorder may be characterized by antigen presenting cells driving abnormal differentiation and expansion of T helper cell subsets. As such, the disease or disorder may comprise an inflammatory disease, a fibrosis disease, infectious disease, and an autoimmune disease. In some embodiments, the disease or disorder does not comprise Graves' disease or thyroid-associated ophthalmopathy.

In some embodiments, the disease or disorder is an inflammatory disease or disorder. Inflammatory diseases are characterized by activation of the immune system in a tissue or an organ to abnormal levels that may lead to abnormal function, dysfunction and/or disease in the tissue or organ. The inflammatory diseases and disorders that may be treated by the methods of the present invention include, but are not limited to, arthritis, rheumatoid arthritis, asthma, chronic obstructive pulmonary disease (COPD), allergic rhinitis, vasculitis (polyarteritis nodosa, temporal arteritis, Wegener's granulomatosis, Takayasu's arteritis, or Behcet's syndrome), inflammatory neuropathy, psoriasis, systemic lupus erythematosus (SLE), chronic thyroiditis, Hashimoto's thyroiditis, Addison's disease, polymyalgia rheumatica, Sjogren's syndrome, Churg-Strauss syndrome, Peyronie's disease, Dupuytren's contracture, mechanical or chemical-induced trauma or a microbial infection.

In some embodiments, the disease or disorder is an autoimmune disease or disorder. Autoimmune diseases and disorders refer to conditions in a subject characterized by cellular, tissue and/or organ injury caused by an immunologic reaction of the subject to its own cells, tissues and/or organs. Autoimmune diseases and disorders that may be treated by the methods of the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, discoid lupus, essential mixed cryoglobulinemia, fibromyalgia-fibromyositis, glomerulonephritis, Guillain-Barre, Hashimoto's thyroiditis, idiopathic pulmonary fibrosis, idiopathic thrombocytopenia purpura (ITP), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatics, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteritis/giant cell arteritis, ulcerative colitis, uveitis, vasculitides such as dermatitis herpetiformis vasculitis, vitiligo, and Wegener's granulomatosis.

In some embodiments, the inflammation and tissue remodeling can arise as a consequence of transplantation and the reaction to heterologous tissues and cells or autologous tissues and cells transplanted in ectopic anatomic locations.

Some autoimmune disorders are also associated with an inflammatory condition. Examples of inflammatory disorders which are also autoimmune disorders that can be prevented, treated, or managed in accordance with the methods of the invention include, but are not limited to, asthma, encephalitis, chronic obstructive pulmonary disease (COPD), allergic disorders, pulmonary fibrosis, undifferentiated spondyloarthropathy, undifferentiated arthropathy, arthritis, inflammatory osteolysis, and chronic inflammation resulting from chronic viral or bacterial infections. Examples of the types of psoriasis which can be treated in accordance with the compositions and methods of the invention include, but are not limited to, plaque psoriasis, pustular psoriasis, erythrodermic psoriasis, guttate psoriasis and inverse psoriasis.

Fibrotic disorders encompass a wide spectrum of clinical entities and are characterized by uncontrolled and progressive accumulation of fibrotic tissue in affected organs (e.g., lung, liver, heart, kidney, and eyes) and tissues causing dysfunction and/or failure. Fibrotic diseases include, but are not limited to: glomerulonephritis; diabetic nephropathy; renal fibrosis; HIV-related nephropathy; transplant gangrene; cirrhosis of any etiology; biliary disorder; liver dysfunction due to infection; pulmonary fibrosis; adult respiratory distress syndrome (ARDS); chronic obstructive pulmonary disease (COPD); fibrosis (IPF); acute lung injury (ALI); lung fibrosis due to infectious or toxic factors; congestive heart failure; dilated cardiomyopathy; myocarditis; vascular stenosis; progressive systemic sclerosis; scleroderma; dermatomyositis; fasciitis; Raynaud's syndrome, rheumatoid arthritis, proliferative vitreoretinopathy; fibrosis associated with ophthalmic surgery; macular degeneration, and some cancers.

Antigen-presenting cells (APC) comprise any cells that can process a protein antigen, break it into peptides, and present it (e.g., in conjunction with class I or class U MHC molecules) on the cell surface where it may interact with appropriate T cell receptors. Classical APCs present the antigen peptide in conjunction with class II MHC molecules, usually, and comprise macrophages, dendritic cells, macrophages, Langerhans cells and B cells. Almost any nucleated cell can act as an APC and present an antigen, usually in conjunction with a class I MHC molecule. In some embodiments, the APC is a fibrocyte. In some embodiments, the APCs comprise CD34+ fibrocytes. In some embodiments, APCs comprise B cell and dendritic cells.

Administration of the at least one IGF-1R inhibitor may attenuate antigen presentation, decrease tissue remodeling, or a combination thereof. The IGF-1R inhibitor may result in a Th17-driven disease to switch to a Th1 driven immune response through IL-23p19 and IL-12p35 expression modulation.

The diseases or disorders may be selected from the group consisting of: neuromyelitis optica spectrum disorder (NMOSD), myasthenia gravis, pemphigus, rheumatoid arthritis, fibrosing or sclerosing cholangitis, pulmonary fibrosis, Peyronie's disease, carpal tunnel syndrome, dermatomyositis, lethal midline granuloma syndrome (LMG), Type I diabetes, ankylosing spondylitis, polymyalgia rheumatica, systemic lupus erythematosus (SLE), and immune thrombocytopenia (ITP) psoriasis, scleroderma, autoimmune hemolytic anemia, Dupuytren's contracture, and combinations thereof.

In some embodiments, the disease or disorder comprises Neuromyelitis optica spectrum disorder (NMOSD), also known as Devic disease. NMOSD is a chronic disorder of the brain and spinal cord dominated by inflammation of the optic nerve (optic neuritis) and inflammation of the spinal cord (myelitis).

Insulin-like growth factor-1 receptor (IGF-1R) inhibitors may be administered for NMOSD with a number of second therapies. The second therapy may be at least one second therapeutic agent (e.g., a corticosteroid, eculizumab, inebilizumab, azathioprine, satralizumab, mycophenolate mofetil, rituximab, antispasticity agents or antispasmodics) or may be a second therapy not connected to administration of another agent (e.g., plasma exchange).

In some embodiments, the disease or disorder comprises myasthenia gravis. Myasthenia gravis is a neuromuscular disorder primarily characterized by muscle weakness and muscle fatigue. The condition may be restricted to certain muscle groups, particularly those of the eyes (ocular myasthenia), or may become more generalized (generalized myasthenia gravis), involving multiple muscle groups. Most individuals with myasthenia gravis develop weakness and drooping of the eyelids (ptosis); weakness of eye muscles, resulting in double vision (diplopia); and excessive muscle fatigue following activity.

Insulin-like growth factor-1 receptor (IGF-1R) inhibitors may be administered for myasthenia gravis with a number of second therapies. The second therapy may be at least one second therapeutic agent (e.g., cholinesterase inhibitors, immunosuppressants, corticosteroids) or may be a second therapy not connected to administration of another agent (e.g., surgery (e.g., thymectomy), plasma exchange).

In some embodiments, the disease or disorder comprises pemphigus. Pemphigus encompasses a group of autoimmune disease which cause blistering of the skin and mucous membranes (mouth, nose, throat, eyes, and genitals).

Insulin-like growth factor-1 receptor (IGF-1R) inhibitors may be administered for pemphigus with a number of second therapies. The second therapy may be at least one second therapeutic agent (e.g., rituximab, immunosuppressants, corticosteroids, intravenous immunoglobulin (WIG)) or may be a second therapy not connected to administration of another agent (e.g., plasma exchange).

In some embodiments, the disease or disorder comprises rheumatoid arthritis. Rheumatoid arthritis is a chronic inflammatory disorder that can affect more than just your joints. In some people, the condition can damage a wide variety of body systems, including the skin, eyes, lungs, heart, and blood vessels

Insulin-like growth factor-1 receptor (IGF-1R) inhibitors may be administered for rheumatoid arthritis with a number of second therapies. The second therapy may be at least one second therapeutic agent (e.g., anti-inflammatory agents, corticosteroids, antirheumatics, monoclonal antibody therapies (e.g., abatacept, adalimumab, etanercept, and the like)) or may be a second therapy not connected to administration of another agent (e.g., synovectomy, joint fusion, joint replacement, physical therapy).

In some embodiments, the disease or disorder comprises fibrosing cholangitis. Fibrosing cholangiopathy, such as primary sclerosing cholangitis (PSC), primary biliary cholangitis (PBC) and biliary atresia (BA), is characterized by fibrosis of small intrahepatic or larger intra- and/or extrahepatic bile ducts with inflammatory cell infiltration and scarring.

Insulin-like growth factor-1 receptor (IGF-1R) inhibitors may be administered for fibrosing cholangitis with a number of second therapies. The second therapy may be at least one second therapeutic agent (e.g., bile acid sequestrants, antibiotics, antihistamines, opioid antagonists, nutritional supplements, bile acids (e.g., ursodeoxycholic acid)) or may be a second therapy not connected to administration of another agent (e.g., balloon dilation or stent for bile duct blockage, liver surgery or transplant).

In some embodiments, the disease or disorder comprises pulmonary fibrosis (PF). Pulmonary fibrosis is a family of more than 200 different lung diseases that present with very similar indications. The PF family of lung diseases falls is a member of a family of interstitial lung diseases (also known as ILD), which includes all of the diseases that have inflammation and/or scarring in the lung. When an interstitial lung disease includes scar tissue in the lung, it is referred to as pulmonary fibrosis.

Insulin-like growth factor-1 receptor (IGF-1R) inhibitors may be administered for pulmonary fibrosis with a number of second therapies. The second therapy may be at least one second therapeutic agent (e.g., anti-fibrotic, anti-inflammatory, corticosteroid, immunosuppressant) or may be a second therapy not connected to administration of another agent (e.g., oxygen therapy, pulmonary rehabilitation).

In some embodiments, the disease or disorder comprises Peyronie's disease. Peyronie's disease is caused by the development of scar tissue inside the penis that results in abnormal curvature of the penis.

Insulin-like growth factor-1 receptor (IGF-1R) inhibitors may be administered for Peyronie's disease with a number of second therapies. The second therapy may be at least one second therapeutic agent (e.g., collagenase, verapamil, interferon-alpha 2B, anti-inflammatory agents) or may be a second therapy not connected to administration of another agent (e.g., mechanical traction, vacuum devices, shockwave therapy, surgery).

In some embodiments, the disease or disorder comprises carpal tunnel syndrome or median nerve compression. Carpal tunnel syndrome is caused by pressure on the median nerve resulting in numbness, tingling and weakness in the hand and arm.

Insulin-like growth factor-1 receptor (IGF-1R) inhibitors may be administered for carpal tunnel syndrome with a number of second therapies. The second therapy may be at least one second therapeutic agent (e.g., anti-inflammatory agents, corticosteroids) or may be a second therapy not connected to administration of another agent (e.g., wrist splinting or surgery).

In some embodiments, the disease or disorder comprises dermatomyositis. Dermatomyositis is an inflammatory disease marked by muscle weakness and a distinctive skin rash. Insulin-like growth factor-1 receptor (IGF-1R) inhibitors may be administered for dermatomyositis with a number of second therapies. The second therapy may be at least one second therapeutic agent (e.g., corticosteroids, rituximab, hydroxychloroquine, IVIG) or may be a second therapy not connected to administration of another agent (e.g., surgery).

In some embodiments, the disease or disorder comprises lethal midline granuloma syndrome. Lethal midline granuloma syndrome comprises a condition whose diagnosis is difficult to be made because of the wide array of related diseases and nonspecific symptoms. Midline destructive lesions of the face were first described in 1897, and later a variety of terms were coined to describe them. A factor that is common to all of such lesions is the development of an ulcerative/vegetative process culminating with destruction of the nasal region, resulting in functional and cosmetic deformity.

Insulin-like growth factor-1 receptor (IGF-1R) inhibitors may be administered for lethal midline granuloma syndrome with a number of second therapies. The second therapy may be at least one second therapeutic agent (e.g., chemotherapeutic agent) or may be a second therapy not connected to administration of another agent (e.g., radiation).

In some embodiments, the disease or disorder comprises type 1 diabetes, once known as juvenile diabetes or insulin-dependent diabetes. Type 1 diabetes is a chronic condition in which the pancreas produces little or no insulin.

Insulin-like growth factor-1 receptor (IGF-1R) inhibitors may be administered for type 1 diabetes with a number of second therapies. The second therapy may be at least one second therapeutic agent (e.g., insulin, high blood pressure medications, aspirin, cholesterol lowering drugs) or may be a second therapy not connected to administration of another agent (e.g., kidney, pancreas, or islet cell transplant).

In some embodiments, the disease or disorder comprises ankylosing spondylitis (AS). Ankylosing spondylitis is a chronic inflammatory disease causing axial arthritis, frequently resulting in inflammatory low back pain early in the disease course, with eventual severe impairment of spinal mobility due to structural changes ultimately leading to spinal fusion. Ankylosing spondylitis is the archetype of a heterogeneous group of arthritides within the rheumatic diseases known formerly as the seronegative spondyloarthropathies but now frequently referred to as spondyloarthritis (SpA). In addition to axial arthritis, ankylosing spondylitis can result in peripheral arthritis, enthesitis, and uveitis, all shared characteristics of the SpA.

Insulin-like growth factor-1 receptor (IGF-1R) inhibitors may be administered for ankylosing spondylitis with a number of second therapies. The second therapy may be at least one second therapeutic agent (e.g., anti-inflammatories, TNF blockers (e.g., adalimumab)) or may be a second therapy not connected to administration of another agent (e.g., physical therapy or surgery).

In some embodiments, the disease or disorder comprises polymyalgia rheumatica. Polymyalgia rheumatica is an inflammatory disorder that causes muscle pain and stiffness, especially in the shoulders and hips.

Insulin-like growth factor-1 receptor (IGF-1R) inhibitors may be administered for polymyalgia rheumatica with a number of second therapies. The second therapy may be at least one second therapeutic agent (e.g., corticosteroids, calcium, vitamin D, methotrexate) or may be a second therapy not connected to administration of another agent (e.g., physical therapy).

In some embodiments, the disease or disorder comprises systemic lupus erythematosus (SLE) or lupus. SLE occurs when your body's immune system attacks your own tissues and organs resulting in inflammation in many different body systems, including joints, skin, kidneys, blood cells, brain, heart, and lungs.

Insulin-like growth factor-1 receptor (IGF-1R) inhibitors may be administered for lupus a number of second therapies. The second therapy may be at least one second therapeutic agent (e.g., anti-inflammatories, antimalarials, corticosteroids, immunosuppressants, rituximab) or may be a second therapy not connected to administration of another agent (e.g., acupuncture).

In some embodiments, the disease or disorder comprises immune thrombocytopenia (ITP), formerly known as idiopathic thrombocytopenic purpura. Immune thrombocytopenia (ITP) is a disorder that can lead to easy or excessive bruising and bleeding due to unusually low levels of platelets. ITP can cause purple bruises, as well as tiny reddish-purple dots that look like a rash.

Insulin-like growth factor-1 receptor (IGF-1R) inhibitors may be administered for ITP a number of second therapies. The second therapy may be at least one second therapeutic agent (e.g., steroids, immunoglobulin, platelet boosting agents (e.g., romiplostim and eltrombopag), rituximab) or may be a second therapy not connected to administration of another agent (e.g., splenectomy, platelet transfusions).

In some embodiments, the disease or disorder comprises psoriasis. Psoriasis is a skin disease characterized by red, itchy scaly patches. Psoriasis goes through cycles, flaring for a few weeks or months, then subsiding for a while or going into remission.

Insulin-like growth factor-1 receptor (IGF-1R) inhibitors may be administered for psoriasis with a number of second therapies. The second therapy may be at least one second therapeutic agent (e.g., corticosteroids, vitamin D or vitamin D analogues, retinoids, calcineurin inhibitors, cyclosporine) or may be a second therapy not connected to administration of another agent (e.g., light therapy).

In some embodiments, the disease or disorder comprises scleroderma. Scleroderma is a rare autoimmune connective tissue disorder characterized by abnormal thickening of the skin. Connective tissue is composed of collagen, which supports and binds other body tissues. There are several types of scleroderma. Some types affect certain, specific parts of the body, while other types can affect the whole body and internal organs (systemic). Scleroderma is also known as progressive systemic sclerosis.

Insulin-like growth factor-1 receptor (IGF-1R) inhibitors may be administered for scleroderma with a number of second therapies. The second therapy may be at least one second therapeutic agent (e.g., rituximab, immunosuppressants, corticosteroids, IVIG) or may be a second therapy not connected to administration of another agent (e.g., plasma exchange).

In some embodiments, the disease or disorder comprises Dupuytren's contracture. Dupuytren's contracture is a hand deformity affecting the layer of tissue that lies under the skin of the palm. Knots of tissue form under the skin, eventually creating a thick cord that can pull one or more fingers into a bent position and prevent complete straightening of the affected fingers.

Insulin-like growth factor-1 receptor (IGF-1R) inhibitors may be administered for s Dupuytren's contracture with a number of second therapies. The second therapy may be at least one second therapeutic agent (e.g., collagenase) or may be a second therapy not connected to administration of another agent (e.g., needling, tissue resection).

In some embodiments, the disease or disorder comprises autoimmune hemolytic anemia. Autoimmune hemolytic anemia develops when the immune system mischaracterizes red blood cells as an unwanted substance and targets them for destruction.

Insulin-like growth factor-1 receptor (IGF-1R) inhibitors may be administered for autoimmune hemolytic anemia with a number of second therapies. The second therapy may be at least one second therapeutic agent (e.g., immunosuppressants, corticosteroids,) or may be a second therapy not connected to administration of another agent (e.g., surgery, blood transfusion).

The insulin-like growth factor-1 receptor (IGF-1R) inhibitors, or compositions thereof, may be administered to the subject by a variety of methods know to those skilled in the art, including without limitation, systemic administration (e.g., oral, rectal, nasal, sublingual, buccal, implants, or parenteral) or topical administration (e.g., dermal, pulmonary, nasal, aural, ocular, liposome delivery systems, or iontophoresis). In some embodiments, administration is directly into the site of disease, inflammation, or fibrosis. For example, in some embodiments, administration is directly into scar tissue or lesion.

The following examples further illustrate the invention but should not be construed as in any way limiting its scope.

### EXAMPLES

### Materials and Methods

Dulbecco's modified Eagle medium (DMEM, cat. #11965) containing 4.5 g/mL D-glucose and L-glutamine, penicillin-streptomycin mixture (cat. #15140), fetal bovine serum (FBS) (cat. #16000-044) and knockout serum replacement (cat. #A3181502) were purchased from Life Technologies (Grand Island, NY). Bovine TSH (bTSH) (cat. #T1614) was from Scripps Laboratories, San Diego, CA. Human CD34+ nucleofection kit (cat. #VPA-1003) was supplied by Lonza, Allendale, N.J. Teprotumumab (RV001) was a gift from River Vision (New York, NY) and Horizon Therapeutics (Lake Forrest, IL). Linsitinib (cat. #A8334) was from APExBIO, Houston, TX. specific TSHR inhibitor ML224 or ANTAG3 (cat #, HY-12381) was from MedChemExpress, Monmouth Junction, NJ. M22 antibody was from Kronus (cat. no. M22-5c/00-690; Star, ID). 5,6-dichlorobenzimidazole (DRB) came from Cayman Chemical (cat. no. 10010302: Ann Arbor, MI). Fixation/Permeabilization Solution Kit (Cat. #554714), 1H7 (Purified Mouse Anti-Human IGF-1R, CD221) Cat. #555998, PE Mouse Anti-Human CD4 (cat. #555347), APC Mouse Anti-Human CD34 (cat. #555824), FITC Mouse Anti-Human HLA-DR (cat. #347363), PE-CF594 Mouse Anti-Human CD184 (CXCR4) cat. #562389, PE-Cy™7 Mouse Anti-Human CD274 (PD-L1) cat. #558017, Alexa Fluor® 700 Mouse Anti-Human CD86 (cat. #561124), V450 Mouse Anti-Human CD80 (cat. #560442), Purified Mouse Anti-Human CD66d/e (cat. #551477), BV605 Mouse Anti-Human IFN-γ (cat. #562974), PerCP-Cy™5.5 Mouse Anti-Human IL-17A (cat. #560799), PE-Cy™7 Mouse Anti-Human CD3 (cat. #557851), PE Mouse Anti-Human CD34 (cat. #555822), anti-mouse IgG1 isotype control PE (cat. no. 555749), and Golgistop (Protein Transport Inhibitor) cat. #554724 were from BD Biosciences (San Diego, CA). Accutase (cat. no. SCR005) was from Merck Millipore (Billerica, MA). Human IL-17A (cat. #D1700) and IFN-gamma (cat. #DIF50) Quantikine ELISA Kits and Recombinant Human IGF-1 (rhIGF-1) (cat. #291-G1) was from R & D systems, (Minneapolis, MN). Pierce Agarose ChIP kit (cat. no. 26156) were from Thermo Fisher Scientific, Rockford, IL. Secrete-Pair Dual Luminescence Assay Kit (cat. #LF033, GeneCopoeia Rockville, MD). Human CD34-Biotin (cat #: 130-092-413), CD34 MicroBead Kit, human (cat. #130-046-702), REAlease CD3 MicroBead Kit, human (cat #: 130-117-038) were from Miltenyi Biotech, Somerville, MA. Phorbol 12-myristate 13-acetate (PMA, cat. #P1585) and ionomycin (cat. #10634) were from Millipore Sigma, Burlington, MA.

Fibrocyte and T cell isolation and cultivation. Peripheral blood mononuclear cells (PBMCs) were obtained from the venous circulation of clinic patients with GD, TAO or from healthy donors after obtaining informed consent. PBMCs were also obtained from Red Cross donor plasmapheresis filters. They were isolated by Ficoll-Histopaque (Sigma-Aldrich, St. Louis, MO) density gradient centrifugation. Fibrocytes were cultivated as described (Bucala, R, et al., (1994) Mol Med 1, 71-81, incorporated herein by reference in its entirety) except that culture substrata remained uncoated with fibronectin, as previously described (Douglas, R. S., et al. (2010) The Journal of clinical endocrinology and metabolism 95, 430-438). Isolated PBMCs (1×10⁶-1×10₇) were inoculated in six-well plates covered with DMEM containing 10% FBS and incubated in 10-14 days. Adherent monolayers (<5% of the starting population) were washed with PBS and detached using accutase (Millipore) treatment, or mechanical scraping following accutase treatment. Culture purity was routinely confirmed by FACS analysis to be >90% fibrocytes by confirming the CD31⁻CD45⁺CD34⁺CXCR4⁺Col I⁺TSFR^{high} phenotype.

Invariably, a small population of tightly adherent T cells were found in the fibrocyte cultures, consistent with the previous findings. Culture viability was routinely verified to be >90%. CD4⁺ T cells were enriched using the CD4 isolation cocktail (Cat #0.130-096-533, Miltenyi Biotec, San Diego, CA) followed by negative selection using a magnetic column (Cat #0.130-042-401) according to manufacturer's protocol. Fibrocytes and isolated T cells were cultured and remained untreated or were incubated with human IgG isotype control (5 μg/ml) or teprotumumab (50 μg/mL) for 7 to 9 days. Single cell suspensions were stained with anti-human CD4, CD3, CD34, MHC-2, CD80, CD86, and PD-L1 (BD Biosciences) for 30 min, rinsed and fixed with CytoPerm/CytoFix (BD). They were then stained with anti-human INFγ and IL-17A for 20 min, rinsed, and subjected to the flow cytometry analysis. Gated CD34⁺CXCR4⁺ fibrocytes and CD4⁺CD3⁺ T cells were analyzed using quadrant settings based on negative isotype staining.

Graves' disease-orbital fibroblast (GD-OF) preparation and cultivtion GD-OF were isolated from orbital adipose/connective tissues removed during surgical orbital decompression surgery, as described ((Smith, T. J., et al., (2002) The Journal of clinical endocrinology and metabolism 87, 385-392). While the fraction of CD34⁺ OF comprising GD-OF strains vary somewhat, the ratios of CD34⁺/CD34⁻ OF are usually 50:50 in the early passages utilized in these studies (passages 2-11). Pure CD34⁺ OF and CD34⁻ OF subsets were generated by staining for 30 m at 4° C. with fluorescein isothiocyanate-conjugated anti-mouse CD34 or its isotype control followed by cytometric cell sorting using a FACSAria III (BD Biosciences) instrument. Cells were sorted under sterile conditions into either CD34⁺ OF, and CD34 OF subsets which were then re-cultured and treated without or with bTSH (5 mU/mL) or the other test compounds, as indicated, for 6 hrs, as indicated.

In vitro studies of CD34⁺ fibrocytes and T cells PBMC and GD-OF monolayers were treated in vitro with human IgG isotype control (5 μg/ml), teprotumumab (50 μg/mL), H7 (5 μg/mL), linsitinib (1 μM), bTSH (5 mIU/mL), M22 (1 μg/mL), or IGF-1 (10 nM) for the intervals indicted in the figure legends in a 37° C., 5% CO₂, humidified incubator until confluent. Brefeldin A (0.66 μl/ml), (Golgi Stop™, BD Biosciences), phorbol 12-myristate 13-acetate (PMA) (50 ng/ml) and ionomycin (1 μg/mL) were added for 5 h. Cells were rinsed with flow cytometry buffer (1×PBS, 0.5% BSA, 0.1% sodium azide) and stained with the relevant monoclonal antibodies.

Studies of circulating CD34⁺ fibrocytes and T cells PBMCs from patients with active, moderate to severe TAO enrolled in a phase 2 trial for teprotumumab were cryopreserved immediately after collection. Venous blood sampling occurred at baseline and at week 24, following 8 infusions at 3 weekly intervals. The post-treatment sample was obtained 3 weeks after the final infusion of active drug or placebo. Samples were thawed and incubated with 10% knockout serum replacement medium for 1h and stained. Rinsed samples were subjected to FACS analysis with a Calibur flow cytometer (BD). Viable cells were gated on the basis of forward light scatter. All samples were analyzed using quadrant settings based on negative staining with isotype control antibodies. Mean fluorescent intensity (MFI) was calculated as the ratio of mean sample to isotype control fluorescence. Data were analyzed with FCS Express software (De Novo Software, Ontario, CA).

ELISAs Media were collected from cultures treated as indicated and analyzed for concentrations of TNF-γ and IL-17A using specific ELISA kits (R&D systems) according to the manufacture's protocol.

IL-12, IL-23, IL-10, HA and BAFF ELISAs. Confluent cultures were treated with nothing or teprotumumab (50 μg/mL) for 5 d, without or with bTSH (5 mU/mL) for the intervals indicated or 72 h. Media and rinsed cell layers were homogenized in 0.1M NaCl and assayed for HA content using an ELISA kit (K-1200) from Echelon Biosciences Inc. (Salt Lake City, UT) according to manufacturer's instructions. A BAFF specific ELISA, purchased from R & D systems, was used in accordance with the manufacturer's instructions.

Real-time PCR Confluent fibrocyte and GD-OF cultures were incubated with bTSH (5 mIU/mL), teprotumumab (50 μg/mL) or linsitinib (1 μM) for the time intervals indicated, washed with PBS and RNA was extracted with the Aurum Total RNA Mini Kit (cat. #732-6820, Bio-Rad, Hercules, CA). Cellular RNA (2 μg) was reverse transcribed using QuantiTect Reverse Transcription kit (cat. #205314, Qiagen (Germantown, MD) and real-time PCR was performed using an Applied Biosystems instrument with QuantiTect SYBR Green PCR kit (cat. no. 204143; Qiagen, Frederick, MD). Primer sequences were as follows.

GAPDH:
SEQ ID NO: 1
5′-TTGCCATCAATGACCCCTTCA-3′ (forward)
SEQ ID NO: 2
5′-GCCCCACTTGATTTTGGA-3′ (reverse)
CD80:
SEQ ID NO: 3
5′-ACGAGTGTGTTGTTCTGAAGTATGA-3′ (forward)
SEQ ID NO: 4
5′-CATTTAATTCTTCTCCATTTTCCAA-3′ (reverse)
CD86:
SEQ ID NO: 5
5′-ACTTCACAATCTTCAG-ATCAAGGAC-3′
(forward)
SEQ ID NO: 6
5′-TGTATAGATGAGCAGGTCAAATTTATG-3′
(reverse)
PD-L1:
SEQ ID NO: 7
5′-TTAGATCCTGAGGAAAACCATACAG-3′ (forward)
SEQ ID NO: 8
5′-TGTGTATCACTTTG-CTTCTTTGAGT-3′
(reverse)
MHC-2:
SEQ ID NO: 9
5′ -CCTTGGGACCTGAGTAGACG-3′ (forward)
SEQ ID NO: 10
5′-CCTTGGGACCTGAGTAGACG-3′ (reverse)
HAS1:
SEQ ID NO: 11
5′ -CGATACTGGGTAGCCTTCAATG-3′(forward)
SEQ ID NO: 12
5′-GGAGGTGTACTTGGTAGCATAACC-3′ (reverse)
HAS2:
SEQ ID NO: 13
5′- GTGTTATACATGTCGAGTTTACTTCC -3′
(forward)
SEQ ID NO: 14
5′- GTC-ATATTGTTGTCCCTTCTTCCGC-3′ (reverse)
BAFF:
SEQ ID NO: 15
5′-GAGAAGCTGCCAGCAGGA-3 (forward)
SEQ ID NO: 16
5′-TCCTGGAGCTGGTGGTTC-3′ (reverse)

PCR values were generated against a standard curve and normalized to respective GAPDH signals.

RNA Pol II ChIP transcription assay Transcriptional assays were performed using the method of Wells and Farnham (Methods (San Diego, Calif.) 26, 48-56(2002). Confluent cultures were treated with human Isotype IgG or teprotumumab (50 μg/mL) in DMEM with 10% FBS for 3 d, rinsed, and shifted to DMEM with 1% FBS for 16 h with the respective additives, and then treated without or with bTSH (5 mU/mL) for 2 h. Cross-linked protein/DNA and isolated chromatin fragments were immunoprecipitated with target antibodies: anti-HLA-DPB1 [EPR11226] (cat. #abl57210), anti-CD80 [EPR22183] (cat. #ab225674), anti-CD86 [EPR21962] (cat. #ab239075), and anti-PD-L1 [EPR19759](cat. #ab213524),) from Abcam (Cambridge, MA) using the Pierce™ Agarose Chip Kit (Thermo Scientific, cat #26156). Eluted fractions were reverse cross-linked and isolated DNA subjected to RT-PCR using EpiTect ChIP qPCR Primer Assay for human HLA-DRA (cat. #GPH1011269(-)02A, HLA-DRB1 (cat. #GPH1024837(-)02A), CD80 (cat. #GPH1023189(-)02A), CD86 (cat. #GPH1009502(-)02A), PD-L1 (cat. #GPH1012902(-)02A, Qiagen. GAPDH served as the housekeeping gene and values were generated according to the protocol provided by the supplier.

Cell transfections, gene promoter assays and siRNA knockdown studies The nucleofection method was utilized to transfect fibrocytes with the U-023 program of the Nucleofector II instrument (Lonza, Walkersville, MD) and the Amaxa human CD34 cell nucleofection kit (cat. no. VPA-1003; Lonza). To assess human gene promoter activities, appropriate fragments described in earlier reports were selected and cloned for MHC II, CD80, CD86, and PD-L1. Confluent fibrocyte monolayers were treated with human Isotype IgG or teprotumumab (50 μg/mL) for 3 d. Cells were transfected with the following target gene promoter fragment sequences supplied by GeneCopoeia (Rockville, MD): MHC-2 (cat. #HPRM49526), −100 to +1429 nt; CD80 (cat. #HPRM45966), −300 to +1350 nt; CD86 (cat. #HPRM46979), −231 to +1272 nt; PD-L1 (cat. #HPRM40139), −237 to +1282 nt; and negative control clone PG04 (cat. #NEG-PG04). After 3-4 d, cells were incubated without or with bTSH (5 mU/mL) for 2 h. Monolayers were harvested and promoter activities measured using the Secrete-Pair Dual Luminescence Assay Kit (cat. #LF033, GeneCopoeia Rockville, MD) according to manufacturer's instructions in an FB12 tube luminometer (Zylux, Huntsville, AL). Values were normalized to internal controls and each experiment was performed at least three times. Small interfering RNAs (siRNA) targeting human IGF-1R ON-TARGET (Cat #L-003012-00-0010)ON-TARGET plus and control (scramble) siRNA (cat. #D-001810-10) and Slit2 (cat #L-019853-00), were from Dharmacon (Lafayette, CO).

Statistics Statistical significance was determined by two-tailed Student's t-test, paired t-test, and ANOVA. Analyses were performed with SAS version 9.4 (Cary, NC).

### Example 1

### Fibrocytes in Culture Constitutively Express MHC 11, CD80, CD86, PD-L1

IGF-1R was initially implicated in the development of thyroid-associated ophthalmopathy (TAO) from experimental findings made entirely in vito, including 1) IGF-1R over-expression by TAO orbital fibroblasts (OF), T cells, and B cells: 2) formation of IGF-1R-thyrotropin receptor (TSHR) physical/functional complexes by orbital fibroblasts, thyroid epithelial cells and thyroid tissues in situ; 3) circulating IGF-1R-targeting autoantibodies in patients with Graves' disease (GD); 4) cell signaling initiated by either IGF-1R or TSHR requires IGF-1R activity.

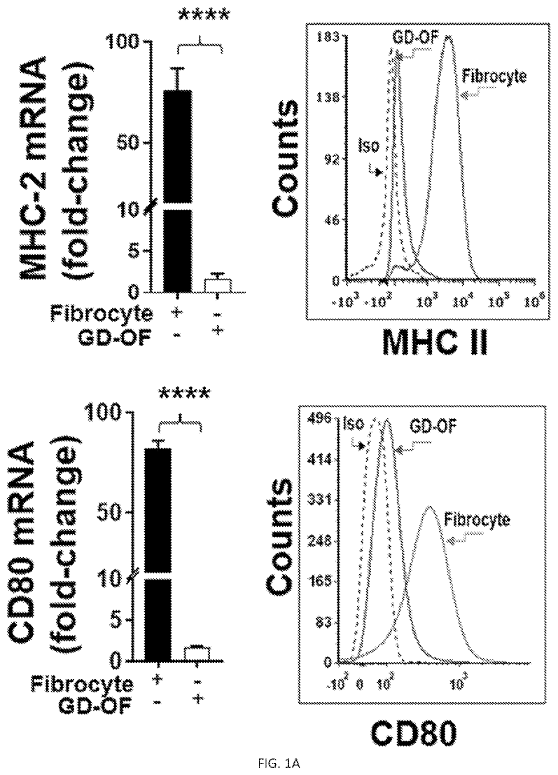

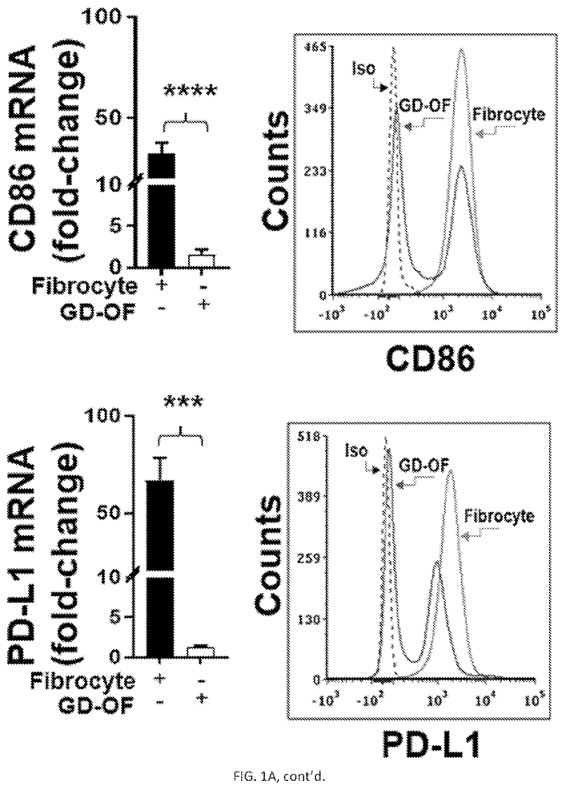

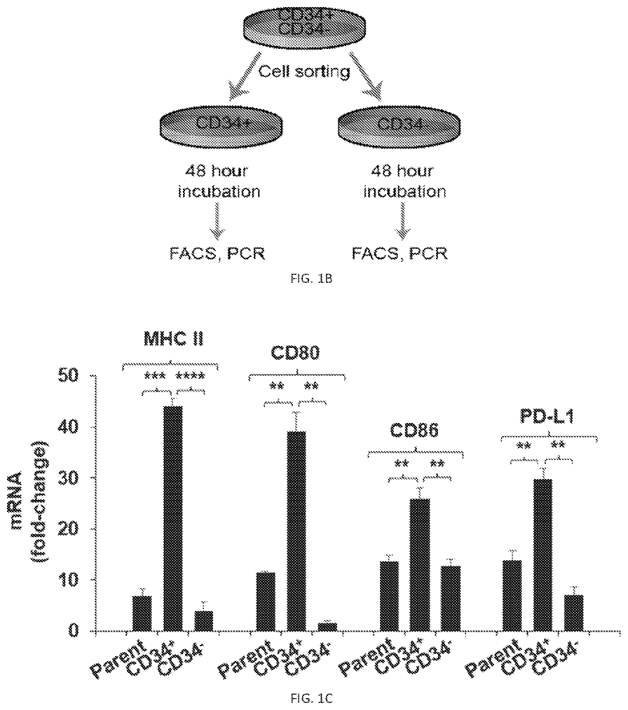

Human fibrocytes cultivated from PBMCs constitutively expressed mRNAs encoding MHC II, CD80, CD86, and PD-L1 (FIG. 1A, left panels). Levels of these transcripts were similar in fibrocytes from patients with TAO and those from healthy donors (data not shown). In contrast, the levels were dramatically higher in fibrocytes than in GD-OF, a substantial fraction of which were their putative derivatives. These disparities between fibrocytes and GD-OF were as follows: MHC II, 94-fold (p<0.001), CD80, 18-fold (p<0.001), CD86, 29-fold (p<0.001), and PD-L1, 56-fold (p<0.001). Cell-surface display of each protein exhibited a similar pattern of relative expression in the two cell-types (FIG. 1A, right panels). To determine whether CD34− OF were involved in the downregulation of the protein expression observed in GD-OF, parental (mixed CD34+ OF and CD34− OF) strains were subjected into cytometric sell-sorting on the basis of CD34 surface display (FIG. 1B), re-cultured for 72 h and the levels of MHC H and B7-encoding transcripts assessed. Data in FIGS. 1C and 1D show the higher levels of these mRNAs and their encoded surface proteins, respectively in pure CD34+ OF compared to either parental GD-OF or pure CD34− OF.

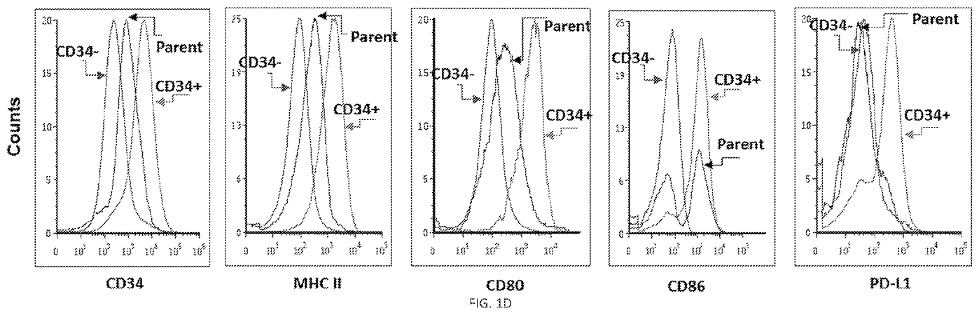

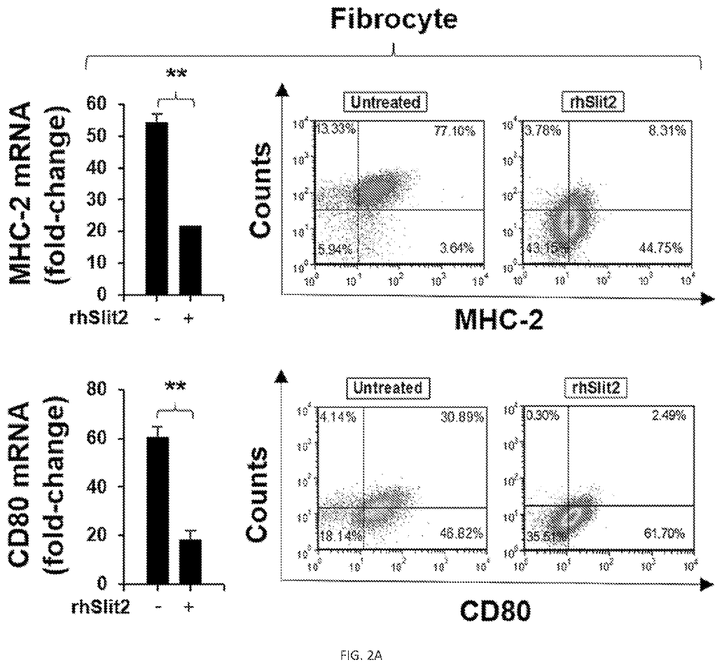

Previously identified divergent levels in fibrocytes compared to GD-OF of “thyroid-specific” autoantigens, autoimmune regulator protein and TAO-implicated cytokines such as IL-23p19 and IL-12p35 result from actions of Slit2, generated by and released from CD34− OF. In the current studies, exogenous rhSlit2 had similar actions in repressing MHC II and B7 expression in CD34+ fibrocytes (FIG. 2A) while knocking-down endogenously expressed Slit2 in GD-OF restored these protein levels toward those in fibrocytes (FIG. 2B). Thus, Slit2 modulated the expression of MHC II and B7 proteins in GD-OF.

### Example 2

### IGF-1R Inhibition Attenuates Fibrocyte-Expressed MHC II and B7 and IL-17A/INFγ Expression in Fibrocyte-Adherent CD4+ T Cells

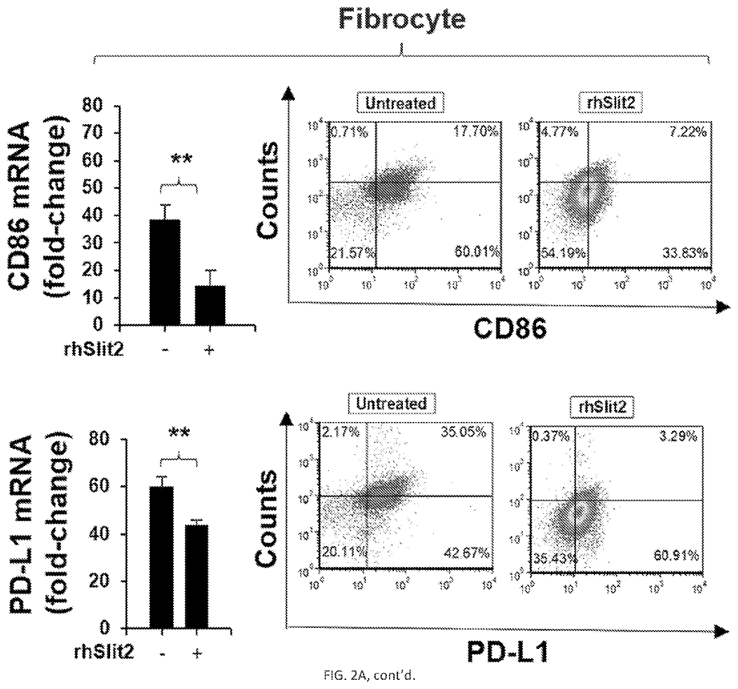

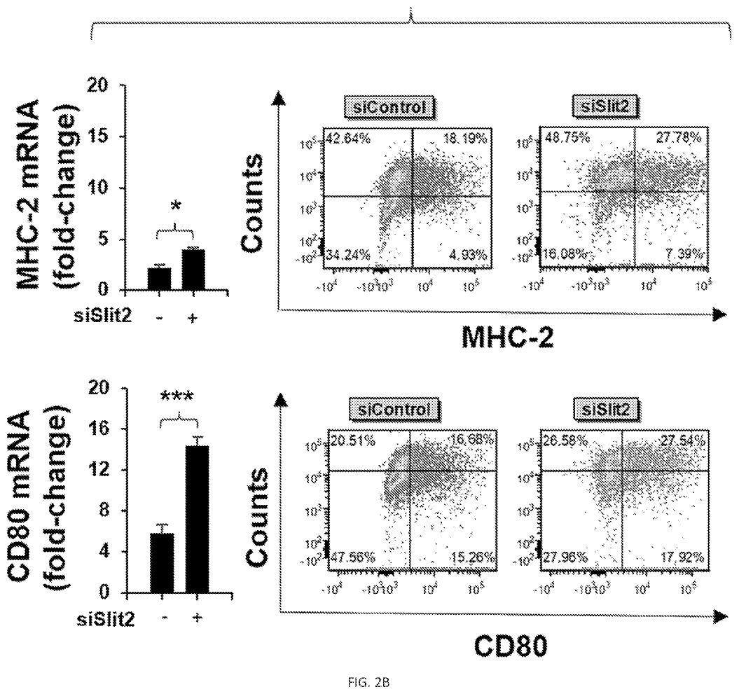

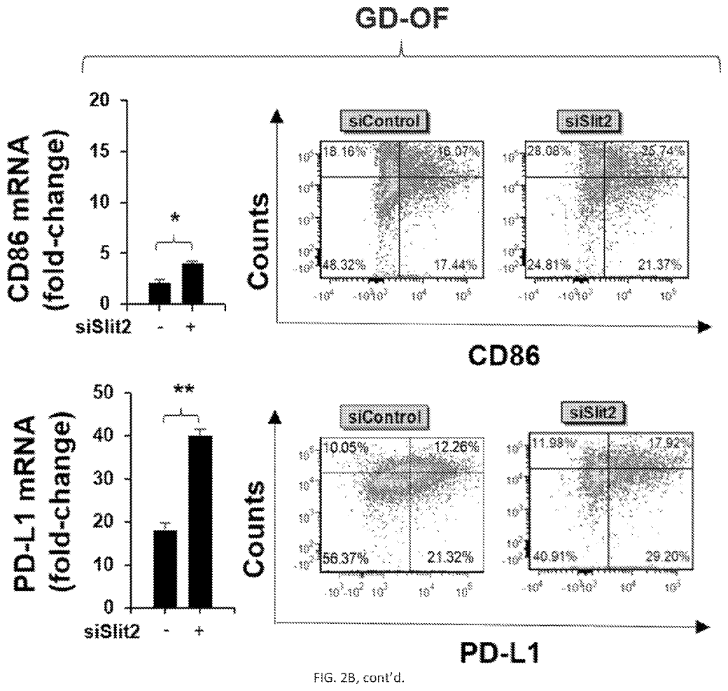

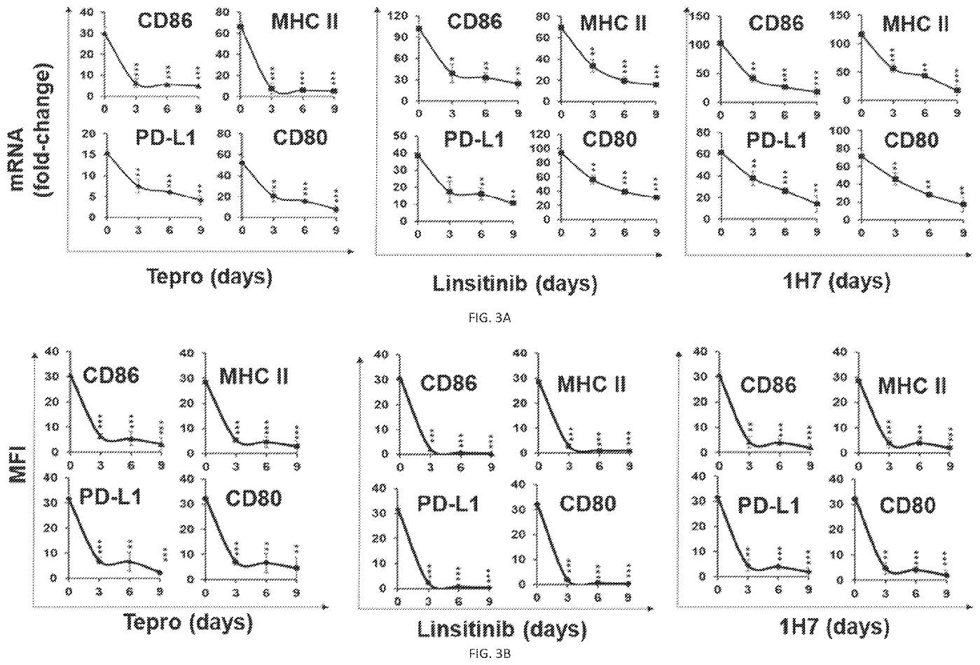

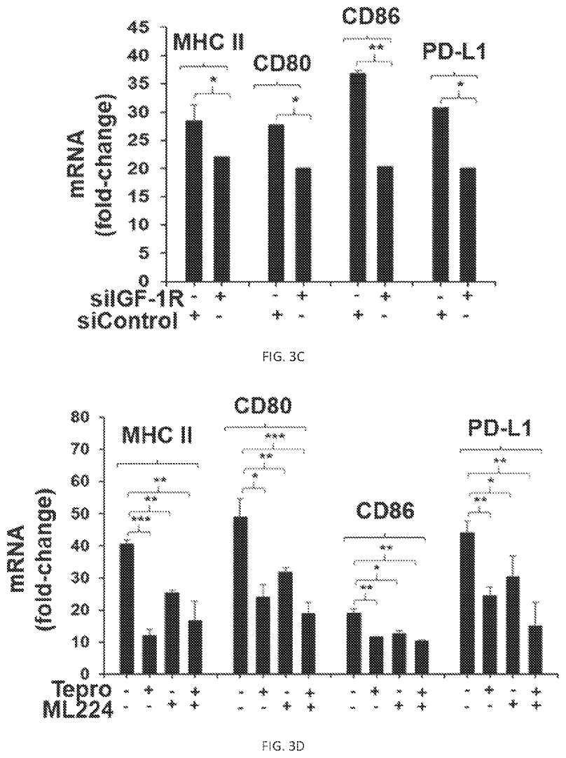

Inhibition of IGF-1R in CD34⁻ fibrocytes reduced the expression of several cytokines. The effects of teprotumumab (50 μg/mL) and those of two other IGF-1R inhibitors, (linsitinib (1 μM) and 1H7 (5 μg/mL)) were determined on MHC II, CD80, CD86, and PD-L1 expression in cultured fibrocytes. Teprotumumab substantially reduced both steady-state mRNA (FIG. 3A) and protein levels (FIG. 3B) of all four proteins in a time-dependent manner extending over a 9 d treatment period. Surface proteins were uniformly reduced to levels of detectability or below at 9 days. Knocking-down IGF-1R with a specific siRNA achieved comparable reductions in expression of these proteins to those of the inhibitors after 5-7 days (FIG. 3C). Effects of teprotumumab on protein expression were compared to that of ML224, a specific inhibitor of TSHR Both were effective as single agents and in combination (FIG. 3D). The three IGF-1R inhibitors also reduced IL-17A and INFγ released into the culture medium of fibrocyte/adherent T cell co-cultures (FIG. 3E). IL-17A and INFγ expression levels were also substantially reduced in the fibrocyte-adherent CD4⁺ T cells examined by cytometry (FIG. 3F upper panel) (IL-17A, control isotype MFI 76.03±14.17 versus teprotumumab MFI 65.17±5.61; INFγ, control MFI 64.01±10.15 versus teprotumumab MFI 26.80±5.61). In contrast, teprotumumab had no such effects on expression cytokine levels in purified, isolated cultured CD3⁺CD4⁺ T cells (IL-17A control isotype MFI 12.69±0.19 versus teprotumumab MEI 12.69±0.31; INFγ control 10.23±0.99 versus teprotumumab MFI 10.32±0.89). This divergence in response to teprotumumab observed in fibrocyte-adherent CD4⁺ T cells and its absence in isolated CD4⁺ T cells strongly suggested that fibrocytes may mediate the drug's effects on T cell expression of IL-17A and INFγ.

### Example 3

### bTSH, M22, and IGF-1 can Induce Expression MHC II, CD80, CD86, and PD-L1 in Fibrocytes, Effects Attenuated by IGF-1R Inhibitors

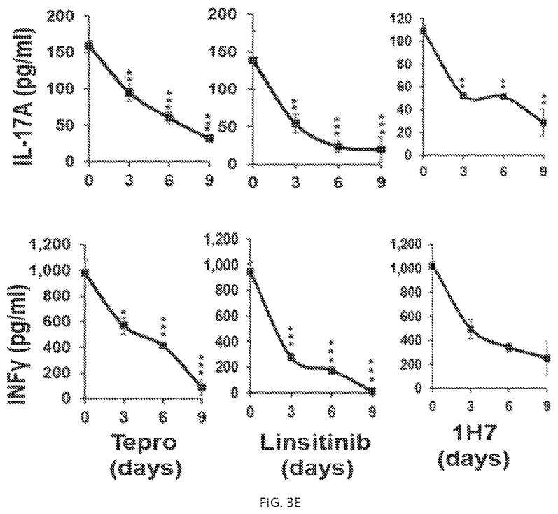

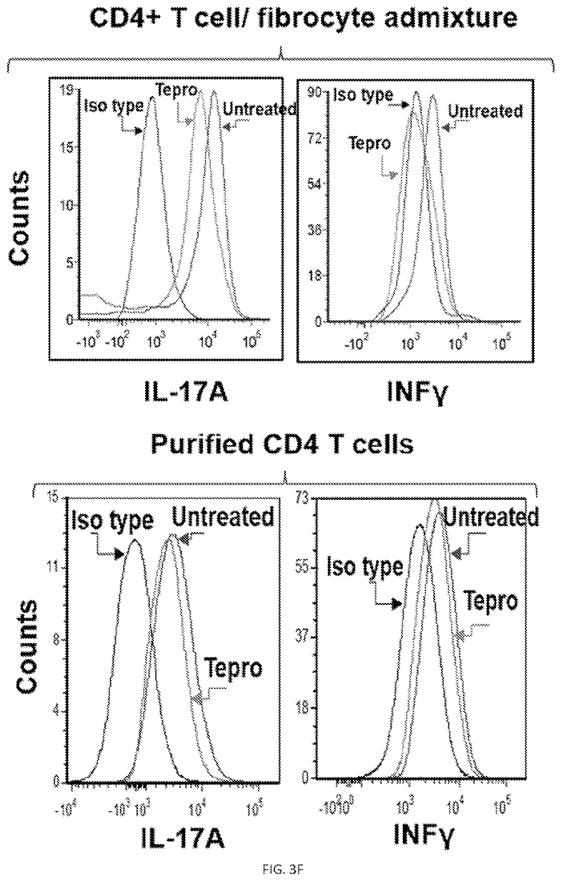

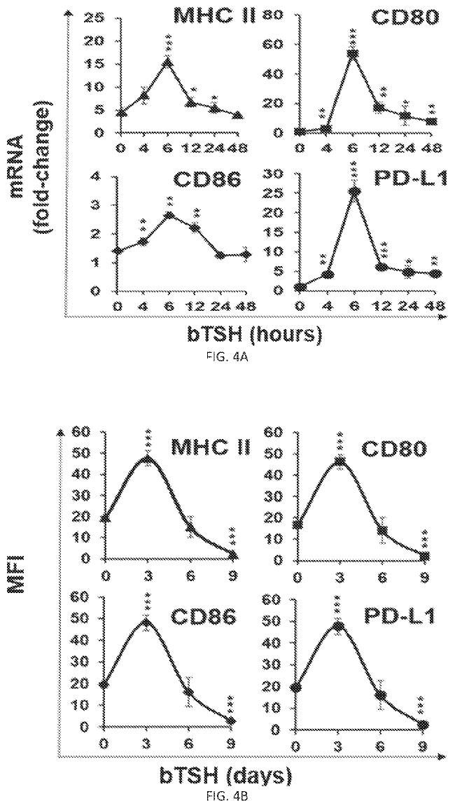

Fibrocytes exhibited a rapid induction by bTSH (5 mIU/mL) of MHC II, CD80, CD86 and PD-L1 mRNAs (FIG. 4A). Each transcript peaked at 6 h (p<0.001 compared to baseline), followed by a steady decay over the following 48 h. Levels of each cell-surface protein were similarly upregulated by approximately 2.5 fold at 72 h in CD34⁺CXCR4⁺ fibrocytes (all p<0.001 versus baseline), followed by a decrease over the course of 9 days, at which time levels of each had fallen below baseline, to the limits of detectability (FIG. 4B). M22, a mAb TSI cloned from a patient with GD, also induced these proteins, reaching a peak at 6 d (FIG. 4C). Responses to rhIGF-1 were considerably less robust than those to bTSH but followed a similar pattern in fibrocytes (FIG. 4D). Teprotumumab, 1H7, and linsitinib blocked the inductions of each mRNA by bTSH and M22 (FIG. 4E) and the respective surface proteins (FIG. 4F), as well. In aggregate, inhibition of IGF-1R activity or its expression reduced basal and induced levels of MHC II and B7 proteins displayed on fibrocytes. The inhibition by teprotumumab was not agent-specific.

### Example 4

### Teprotumumab Attenuates Constitutive and TSH-Induced MHC H, CD80, CD86, and PD-L1 Gene Promoter and Transcriptional Activities and Hastens mRNA Decay

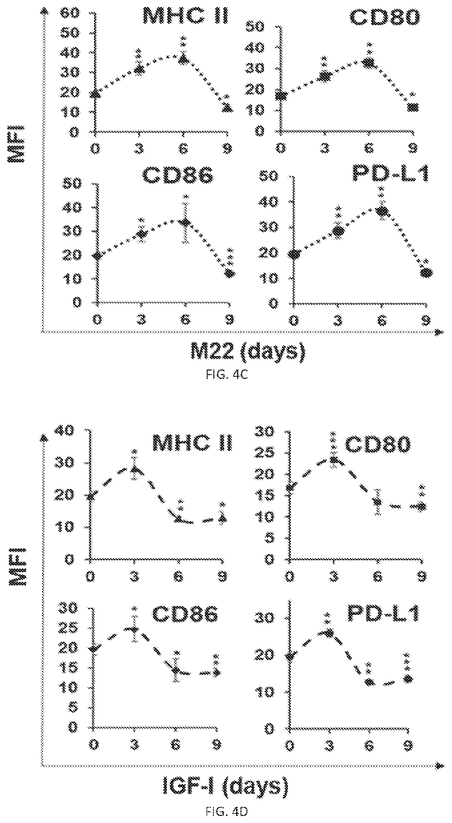

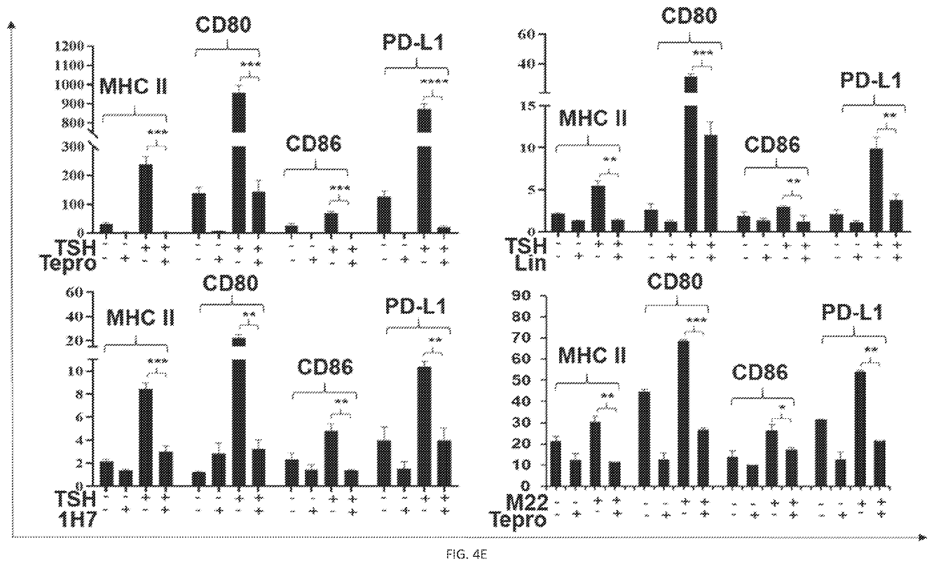

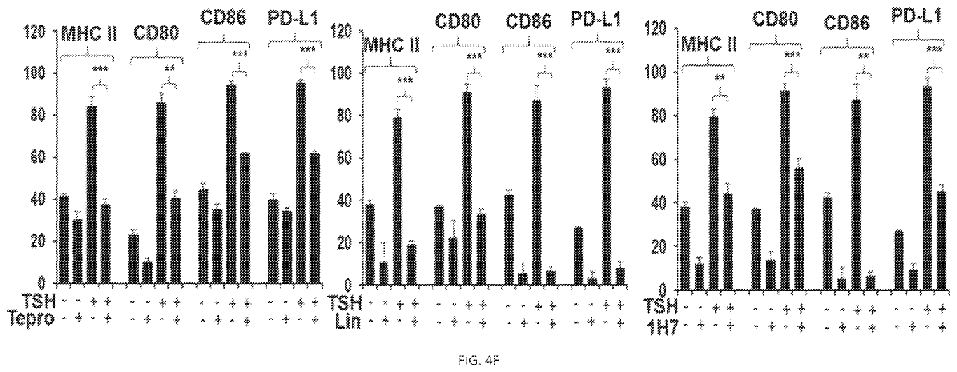

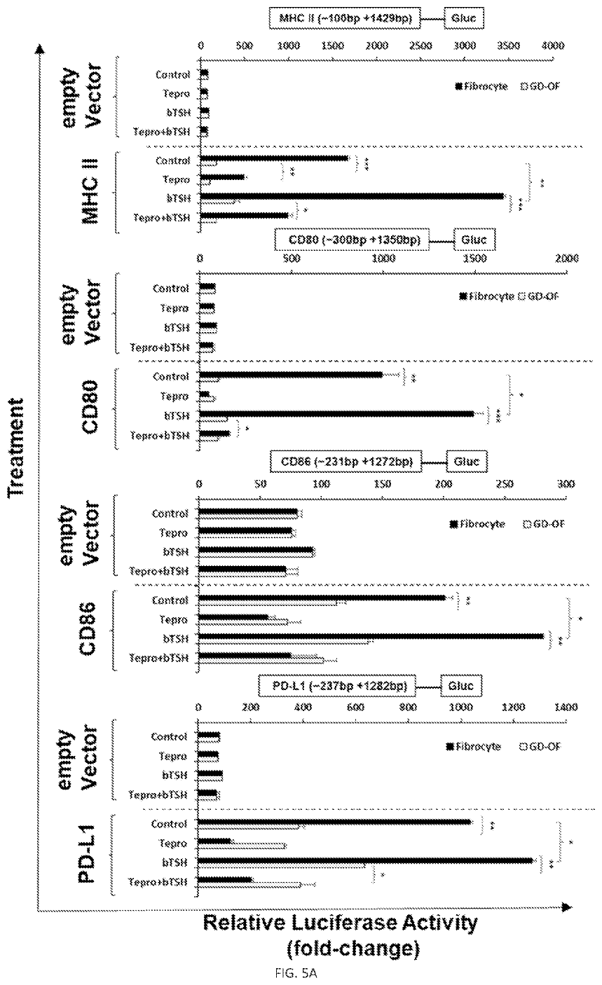

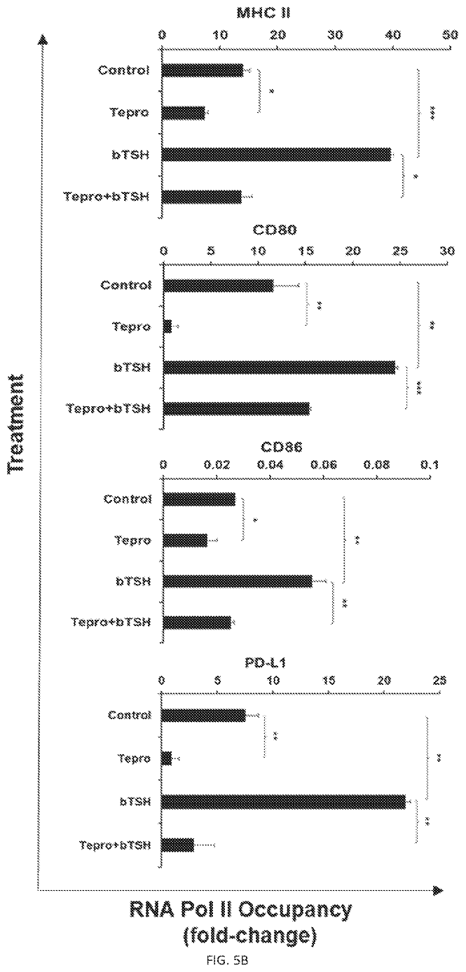

Compared with their levels in GD-OF, activities of gene promoter fragments from MHC U, CD80, CD86, and PD-L1 transfected into fibrocytes exhibited substantial greater reporter activity (FIG. 5A). bTSH (2 h) upregulated these activities while treatment with teprotumumab (50 μg/mL) (3 days) downregulated them. Further, teprotumumab attenuated the inductions of bTSH of each fragment. Gene transcription rates, as assessed by Pol II Chip assays, were similarly induced by bTSH while teprotumumab attenuated both basal and bTSH-induced transcription of these four genes (FIG. 5B). The mRNA stabilities encoding MHC II, CD80, CD86, and PD-L1 were enhanced by bTSH while teprotumumab accelerated their decline (FIG. 5C). The effects of each impact both the contour of mRNA decay, including initial velocities and the final abundances of transcript at the final time-point of the study (12 hrs). Thus, the effects of bTSH and teprotumumab on the expression of these genes appeared to be mediated through both effects on gene transcription and mRNA stabilities.

### Example 5

### Comparisons of Responses to Teprotumumab In Vitro in Fibrocytes from Healthy Donors and Those with TAO

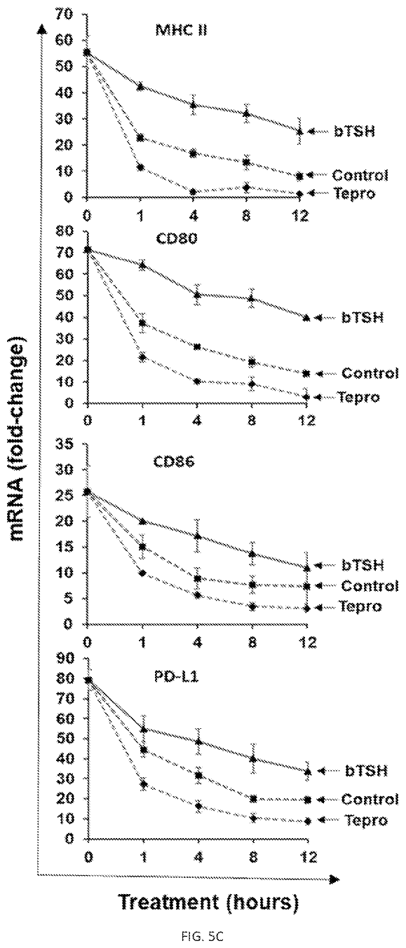

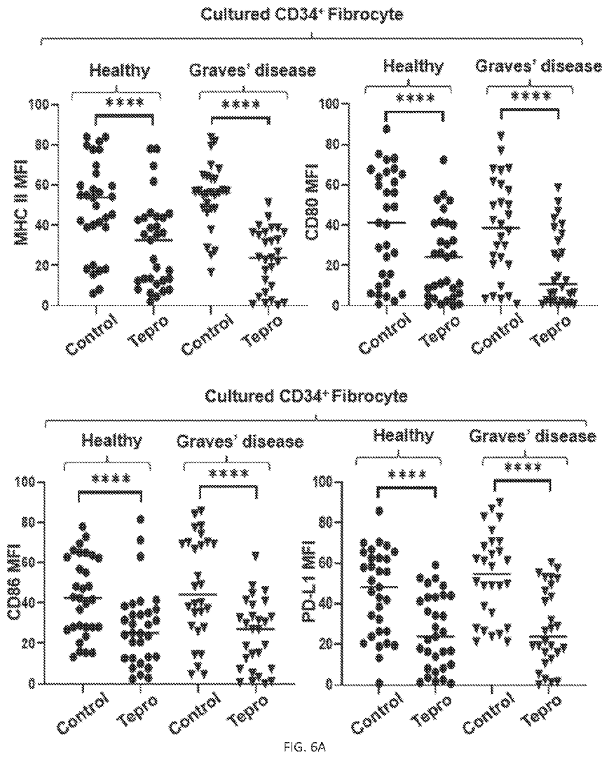

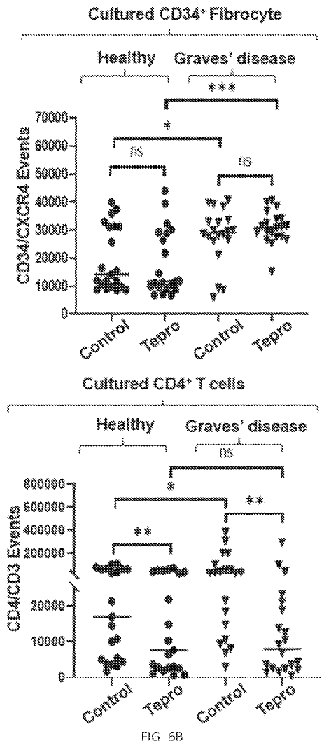

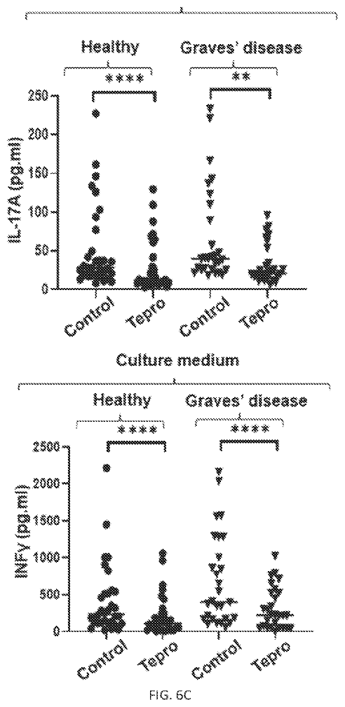

Comparisons of responses to teprotumumab in cultured fibrocytes from patients with GD (n=28) and healthy donors (n=31) revealed similar patterns of inhibition (FIG. 6A). Reductions in levels of MHC II (57%), CD80 (52%), CD86 (49%) and PD-L1 (47%) cell-surface proteins (all p<0.0001) in GD cells treated with teprotumumab were similar to those in healthy control fibrocytes. In contrast, the abundance of CD34⁺ fibrocytes cultivated from PBMCs from GD patients was substantially greater than that from healthy controls (FIG. 6B, upper panel, p<0.01). This increase was consistent with the earlier reports; however, the frequency of CD34⁺ fibrocytes is unaffected by teprotumumab. The abundance of fibrocyte-adherent CD3⁺CD4⁺ T cells was similar in cultures from both donor cohorts (FIG. 6B lower panel) while teprotumumab substantially reduced abundance of adherent CD3⁺CD4⁺ T cells (both p<0.01). Teprotumumab reduced levels of IL-17A and INFγ released into the culture media (FIG. 6C, 52% and 54%, respectively, both p<0.001).

### Example 6

### Teprotumumab and MHC 11 and B7 Protein Expression in Fibrocytes In Vivo in Patients with TAO

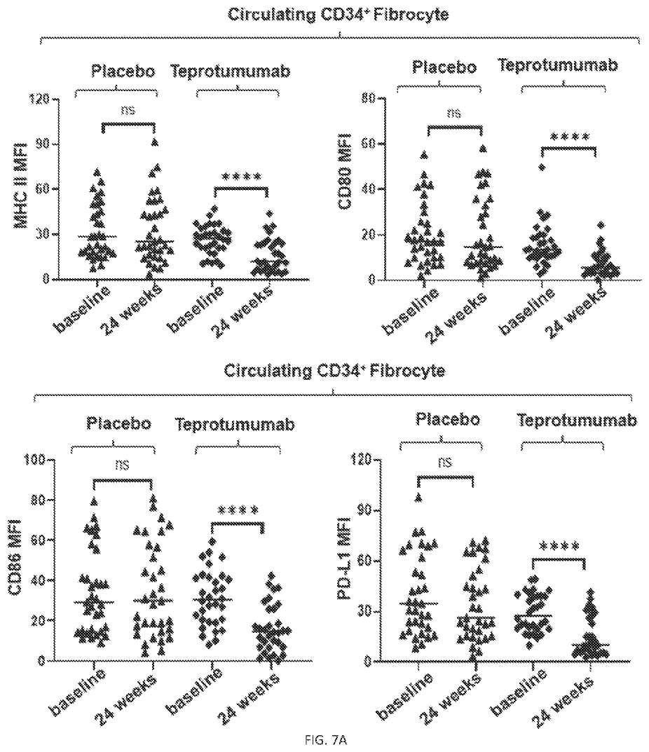

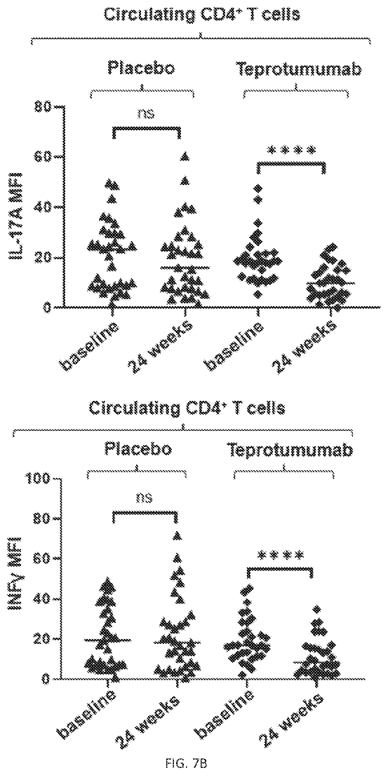

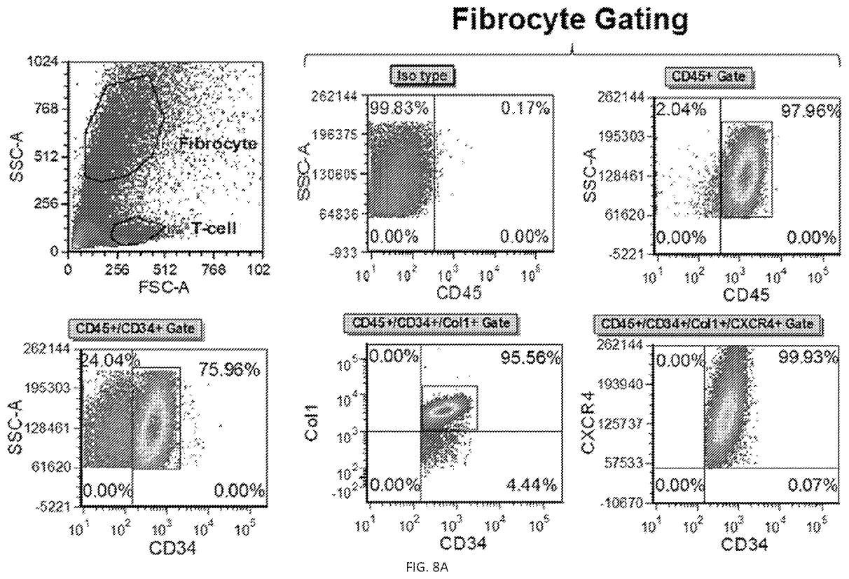

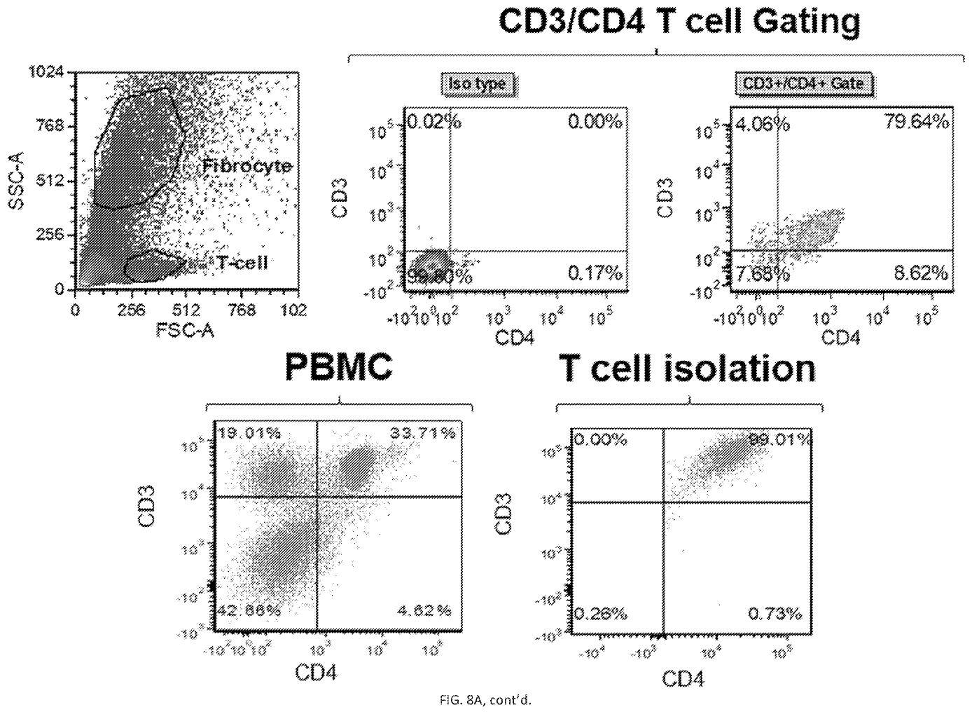

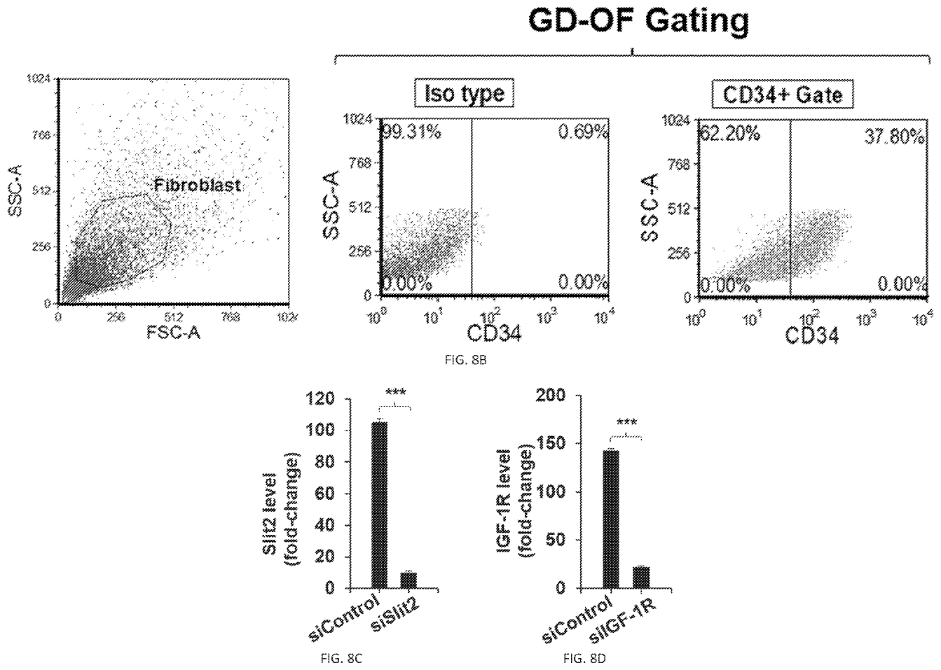

Analysis of circulating PBMCs collected during a randomized phase 2 clinical trial of moderate to severe, active TAO prior to (baseline) and following treatment over 24 weeks with either teprotumumab (20 mg/Kg body weight) or placebo utilized multi-parameter flow cytometry. Teprotumumab reduced in CD34+CXCR4+ fibrocytes the expression levels after a 24-week treatment period compared to baseline of MHC-II (40%, p<0.0001), CD80 (54%, p<0.0001), CD86 (47%, p<0.0001), and PD-L1 (47%, p<0.0001) compared to an absence of changes from baseline in the placebo group (n.s.) (FIG. 7A). Active drug treatment also resulted in reduced expression levels of INFγ (41%, p<0.0001) and IL-17A (47%, p<0.0001) in CD4+ T cells without changes from baseline in the placebo group (FIG. 7B), similar responses to those found in vitro in fibrocyte-adherent CD34+/CD4+ T cells (FIG. 6C). Thus, teprotumumab reduced levels of MHC II and multiple B7 costimulatory molecules in fibrocytes both in vitro and in vivo. Attenuation of these molecules may be associated with reduced INFγ and IL-17A expression in CD4+ T cells in patients with TAO.

### Example 7

### IGF-1R Inhibitors and IL-23p19 and IL-12p35 Expression

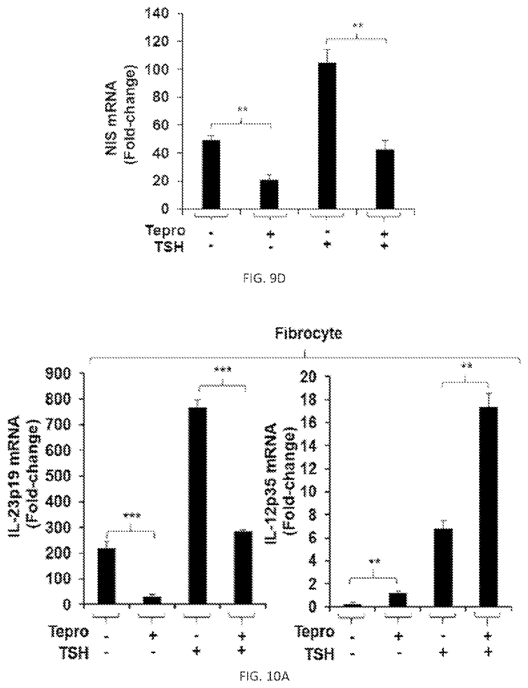

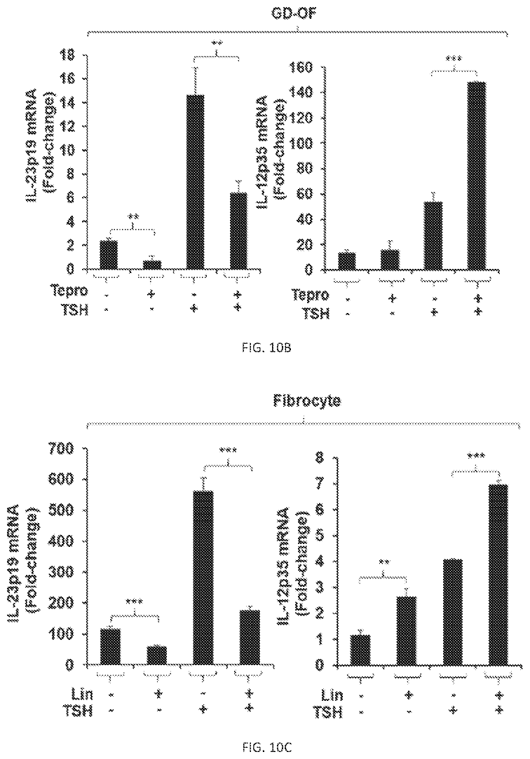

IL-12 and IL-23 are two key cytokines determining immune responses, in part through the polarization and expansion of T cells to the Th1 and Th17 phenotype, respectively. They are differentially expressed in fibrocytes and GD-OF, apparently the consequence of divergent actions of endogenously-expressed Slit2 in the CD34− OF subset. In GD-OF, the down-regulatory effects of CD34− OF have been shown to be the consequence, at least in part, of Slit2. Analysis of IGF-1R inhibition on IL-23p19 and TL-12p35 expression was analyzed as shown in FIG. 10. Teprotumumab and linsitinib both show reciprocal effects on IL-23p19 and IL-12p35 expression, increasing IL-12p35 expression while decreasing IL-23p19 expression.

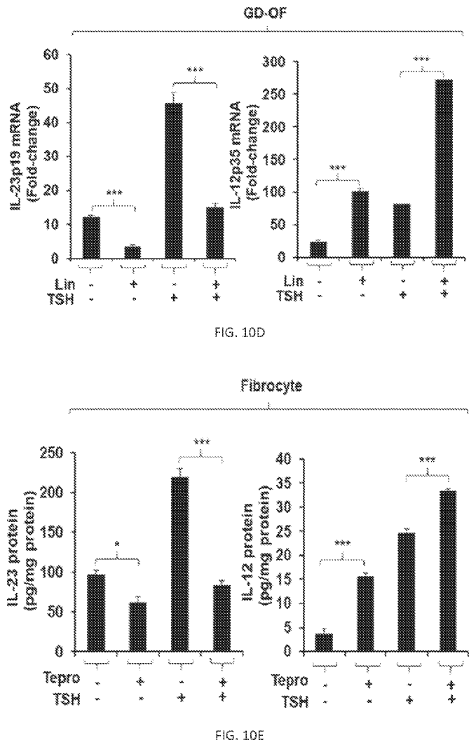

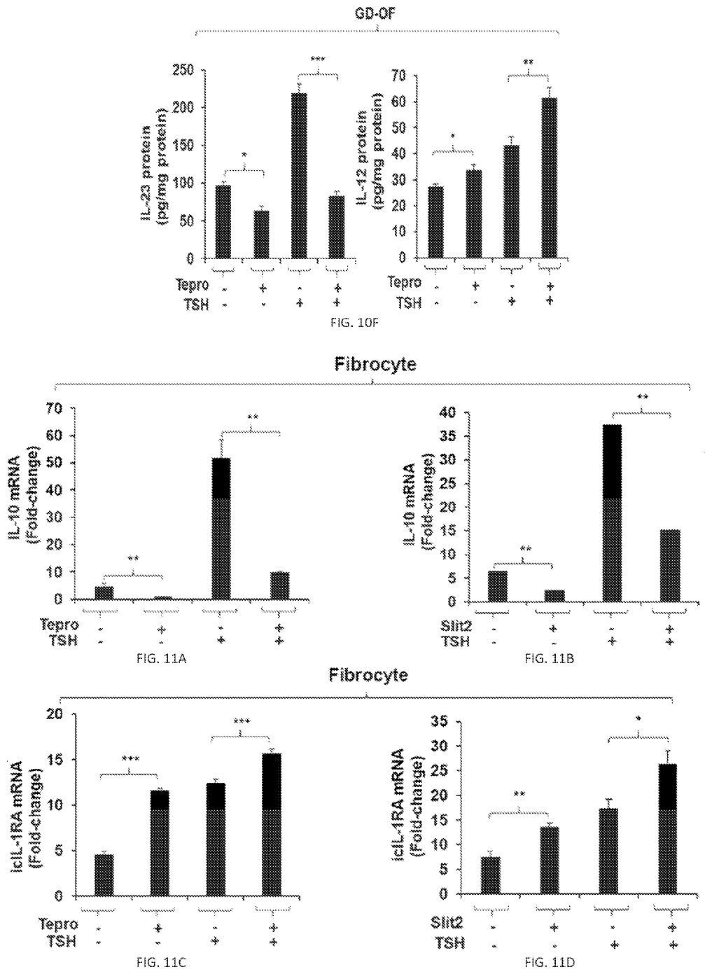

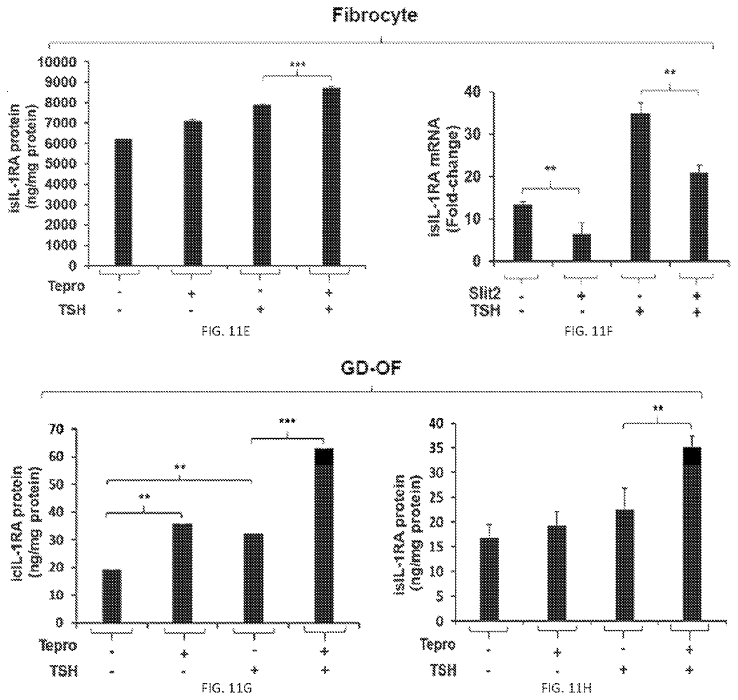

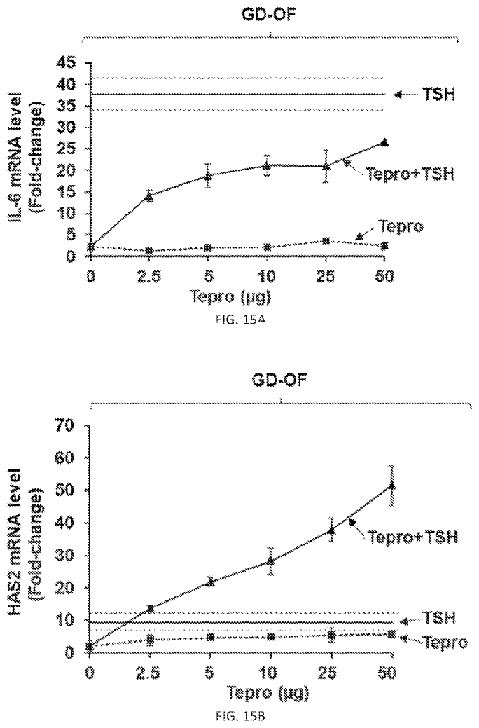

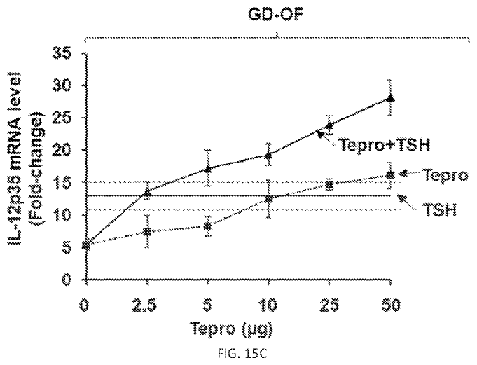

Teprotumumab inhibited basal IL-23p19 expression and induction by bTSH in both fibrocytes (FIG. 10A) and GD-OF (FIG. 10B). In contrast, the IGF-1R inhibitor enhances IL-12p35 expression and induction by bTSH in both cell types. The effects of teprotumumab are dose-dependent (FIG. 15). The impact of linsitinib (1 μM), a small molecule IGF-1R inhibitor, was congruent with that of teprotumumab, and similarly discordant with regard to IL-23p19 and IL-12p35 in these cell types (FIGS. 10C and 10D). These findings suggested that IGF-1R may play a role in maintaining the balance between IL-23 and IL-12 generation in both fibrocytes and GD-OF, effects that resemble those of the Slit2/ROBO1 pathway. In contrast to IL-12 and IL-23, cytokines generally enhance immune responses, IL-10 most frequently inhibits inflammation. IL-10 attenuates immune responses as a component of the T cell regulatory mechanism. Fibrocytes express IL-10 mRNA, as demonstrated in FIG. 11A. bTSH induced IL-10 by 11-fold while both teprotumumab (FIG. 11A) and Slit2 (FIG. 11B) downregulated both basal and bTSH-provoked expression. In contrast, teprotumumab (FIG. 11C) and Slit2 (FIG. 11D) upregulated basal and bTSH-dependent icIL-1RA and sIL-1RA mRNA levels in fibrocytes. The effects of teprotumumab on icIL-1RA protein levels were minimal (FIG. 11E) while Slit2 attenuated sIL-1RA expression in fibrocytes (FIG. 11F). Teprotumumab upregulated both icIL-1RA (FIG. 11G) and sIL-1RA (FIG. 11H) protein levels in GD-OF.

### Example 8

### Teprotumumab and Thyroid Autoantigen Expression in Fibrocytes

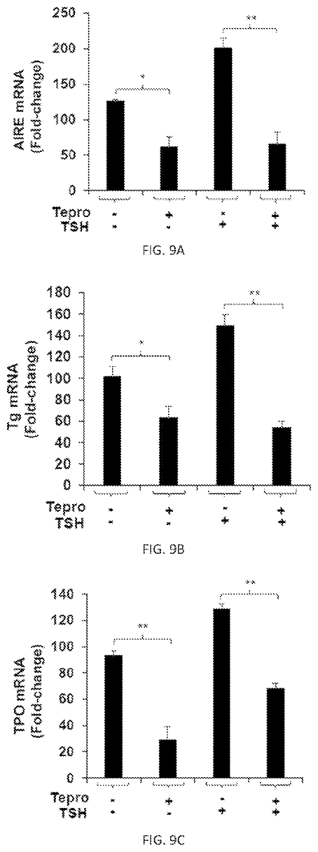

Fibrocytes express relatively high levels of functional TSHR, Tg, NIS, and TPO. Expression of those proteins is dependent on the AIRE protein, which in fibrocytes, is inducible by TSH. Further, Slit2, a neural axon repellent generated by CD34− OF, attenuates the expression of these proteins. Teprotumumab (50 μg/mL) for 7-9 days inhibited the basal expression and induction by bTSH (5 mIU/mL for 6 h) of AIRE (FIG. 9A), Tg (FIG. 9B), TPO (FIG. 9C) and NIS (FIG. 9D) in cultured fibrocytes compared with an isotype control mAb.

### Example 9

### Teprotumumab Inhibits Basal BAFF Expression and Induction by bTSH

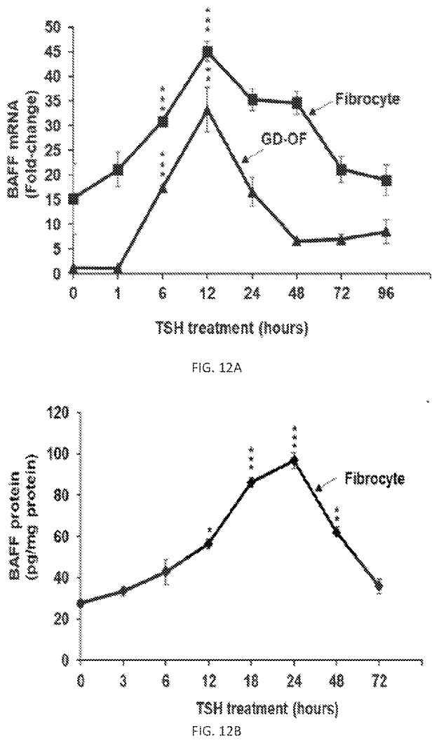

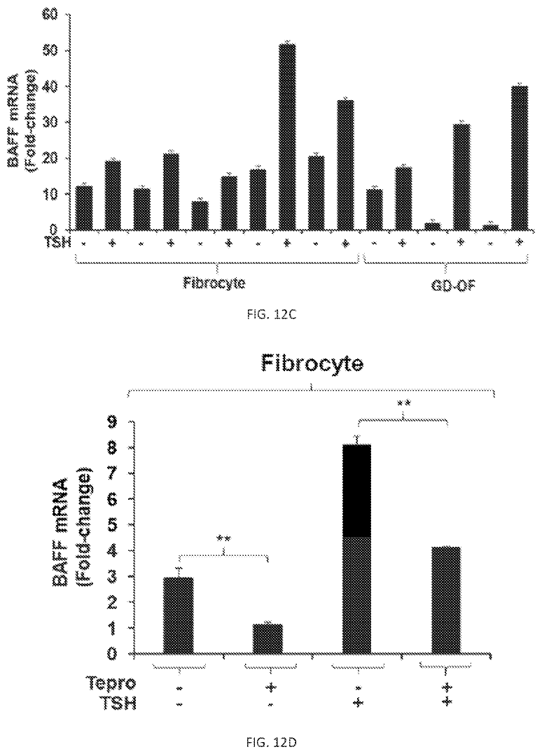

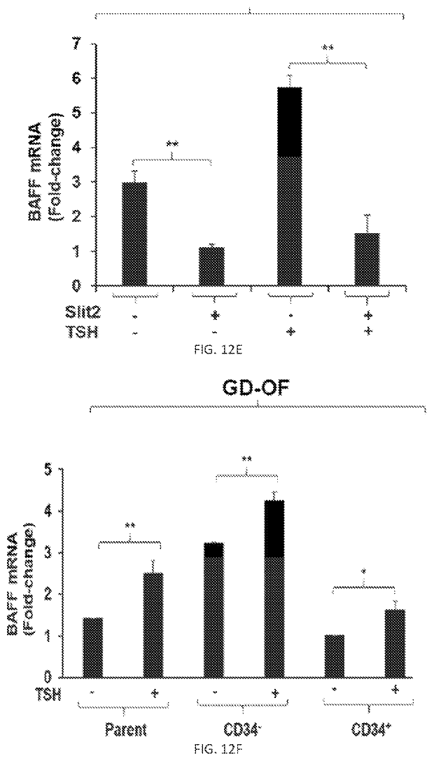

BAFF and its pathway exert important influence on B cell survival. Since its discovery, BAFF has been the focus of developmental therapeutic targeting of autoreactive B cells. Since autoantibody generation appears integral to GD and TAO, it was determined whether fibrocytes and GD-OF generate BAFF. Constitutive BAFF mRNA levels were substantially higher in fibrocytes than in GD-OF (FIG. 12A, 1.4-fold, p<0.05). The transcript was time-dependently induced by bTSH in both cell types, peaking at 12 h and then declining. By 96 h, levels in fibrocytes returned to baseline while those in GD-OF remained elevated. BAFF protein levels were similarly induced in fibrocytes peaking at 24 h and returning to baseline levels by 72 h (FIG. 12B). Teprotumumab attenuated bTSH induction of BAFF (FIG. 12D), effects similar to those of Slit2 (FIG. 12E). To determine whether the relatively low BAFF expression levels in GD-OF compared to fibrocytes were the consequence of factors from CD34− OF, parental strains of GD-OF containing a mixture of CD34+ OF and CD34− OF were subjected to cytometric cell sorting. As the data in FIG. 12F indicates, both basal and bTSH-induced BAFF expression in the pure CD34. OF subset were considerably higher than that in either parental cultures or pure CD34− OF.

### Example 10

### Teprotumumab Enhanced Hyaluronan (HA) Synthesis in GD-OF and Fibrocytes

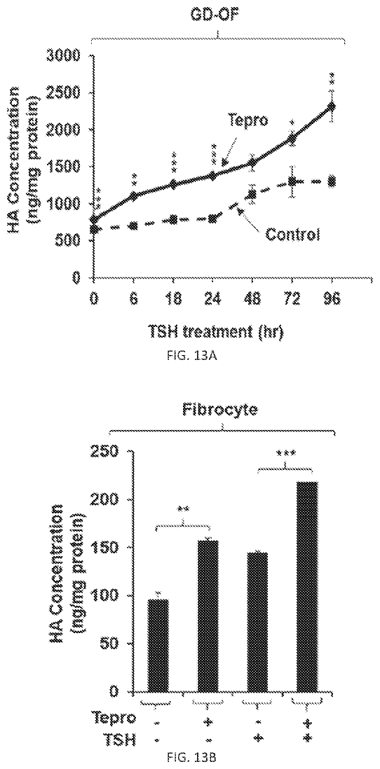

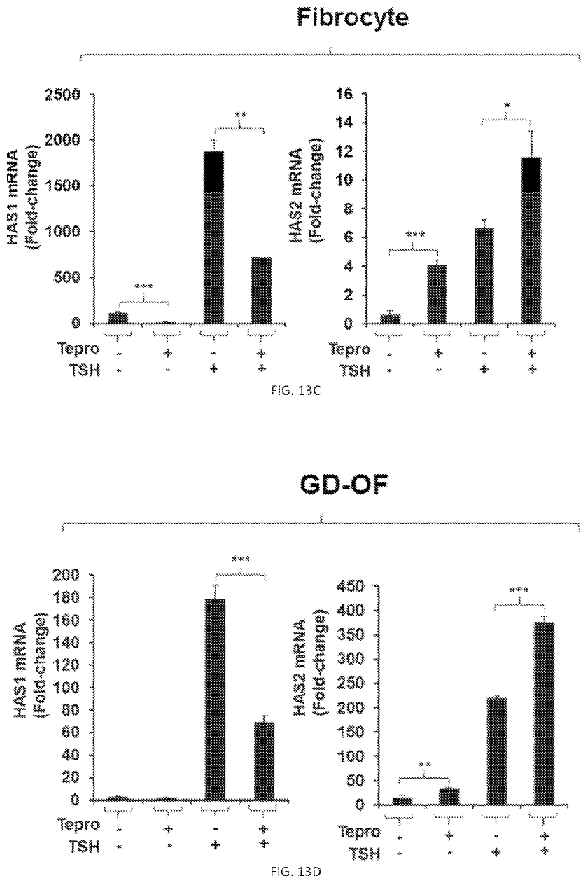

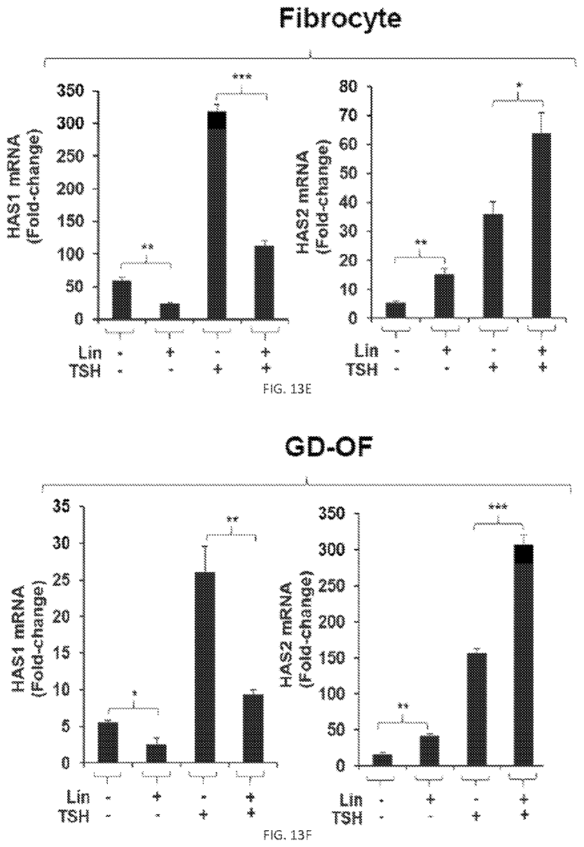

A major component of tissue remodeling in TAO is HA accumulation in the orbit and upper face. Among the clinical responses to teprotumumab in TAO is the dramatic reduction in proptosis accompanied by reductions in orbital connective tissue volumes. Since this increased volume can be attributed, at least in part, to disordered HA accumulation, effects of teprotumumab on HA generation were assessed in fibrocytes and GD-OF. HA synthesis was substantially greater in GD-OF than in fibrocytes. Surprisingly, teprotumumab enhanced the effects of bTSH on HA synthesis in GD-OF over 96 h, the duration of the study (FIG. 13A). The synergism between bTSH and teprotumumab was also apparent in fibrocytes (FIG. 13B). Teprotumumab inhibited both basal and bTSH-provoked expression of HAS1 in fibrocytes and GD-OF (FIGS. 13C and 13D, respectively). In contrast, teprotumumab induced HAS2 expression, either as a single agent or in combination with bTSH in both cell types. The effects of teprotumumab on HAS1 and HAS2 expression are nearly identical to those of linsitinib (1 μM) (FIGS. 13E and 13F). HA production in fibrocytes and GD-OF is primarily the consequence of HAS2 expression and activity. Thus, teprotumumab exerts a small, direct, stimulatory effect on HA production in both fibrocytes and GD-OF through modest HAS2 induction. These effects are similar to those of linsitinib. IGF-1R inhibition can increase in HAS2 levels and HA production in these cells.

### Example 11

### Teprotumumab and Slit2/ROBO1 Pathway

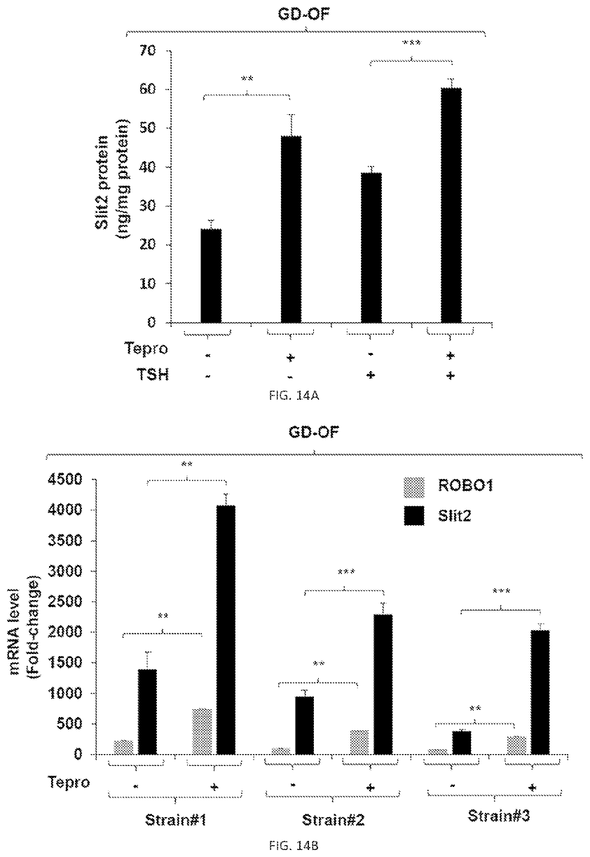

Slit2 exerts a powerful regulatory influence on fibrocyte differentiation and phenotype. Moreover, these effects are mediated through its cognate membrane receptor, roundabout 1 (ROBO1). Because many of the effects exhibited by teprotumumab on fibrocytes resemble those of Slit2, it was examined whether the drug might be influencing Slit2 and ROBO1 expression levels in GD-OF. Teprotumumab induced the expression of both Slit2 mRNA and ROBO1 mRNA in three strains of GD-OF, each from a different donor (FIG. 14A). Further, the drug increased Slit2 protein synthesis in these cells (FIG. 14B).

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating or preventing a disease or disorder in a subject comprising administering to the subject a therapeutically or prophylactically effective amount of at least one insulin-like growth factor-1 receptor (IGF-1R) inhibitor or a composition thereof,
wherein the disease or disorder is characterized by antigen presenting cells driving abnormal differentiation and expansion of T helper cells, and
wherein the disease or disorder does not comprise Graves' disease or thyroid-associated ophthalmopathy.

2. The method of claim 1, wherein the antigen presenting cells comprise CD34+ fibrocytes.

3. The method of claim 1, wherein the T helper cells comprise Th1 and Th17 polarized T cells.

4. The method of claim 1, wherein administration of the at least one IGF-1R inhibitor attenuates antigen presentation, decreases tissue remodeling, or a combination thereof.

5. The method of claim 1, wherein administration of the at least one IGF-1R inhibitor increases HAS2 expression, hyaluronan production, or a combination thereof.

6. The method of claim 1, wherein administration of the at least one IGF-1R inhibitor attenuates the level of one or more of: autoimmune regulator protein, thyroglobulin, sodium iodide symporter, thyroperoxidase, IL-10 and B-cell activating factor.

7. The method of claim 1, wherein the disease or disorder is an inflammatory, fibrosis, infectious, or mechanical/chemical-induced and autoimmune disease.

8. The method of claim 1, wherein the disease or disorder is selected from the group consisting of: neuromyelitis optica spectrum disorder (NMOSD), myasthenia gravis, pemphigus, rheumatoid arthritis, fibrosing or sclerosing cholangitis, pulmonary fibrosis, Peyronie's disease, carpal tunnel syndrome, Dupuytren's contracture, dermatomyositis, lethal midline granuloma syndrome (LMG), Type I diabetes, ankylosing spondylitis, polymyalgia rheumatica, systemic lupus erythematosus (SLE), and immune thrombocytopenia (ITP) psoriasis, scleroderma, mechanical or chemical induced tissue trauma, autoimmune hemolytic anemia, and combinations thereof.

9. The method of claim 1, wherein the IGF-1R inhibitor comprises an antibody against IGF-1R or an IGF-1R tyrosine kinase inhibitor.

10. The method of claim 1, wherein the IGF-1R inhibitor comprises teprotumumab, linsitinib, IGF-1R Antibody 1H7, or a combination thereof.

11. The method of claim 1, further comprising administration of at least one additional therapeutic agent.

12. The method of claim 11, wherein the at least one additional therapeutic agent comprises a corticosteroid, an immunosuppressant, rituximab, an anti-inflammatory agent, an antibiotic, an opioid antagonist, a vitamin or nutritional supplement, or any combination thereof.


SEQUENCE LISTING (16 sequences)

SEQ ID NO: 1 - DNA, 21 bp, artificial sequence
  Synthetic
ttgccatcaatgaccccttca

SEQ ID NO: 2 - DNA, 18 bp, artificial sequence
  Synthetic
gccccacttgattttgga

SEQ ID NO: 3 - DNA, 25 bp, artificial sequence
  Synthetic
acgagtgtgttgttctgaagtatga

SEQ ID NO: 4 - DNA, 25 bp, artificial sequence
  Synthetic
catttaattcttctccattttccaa

SEQ ID NO: 5 - DNA, 25 bp, artificial sequence
  Synthetic
acttcacaatcttcagatcaaggac

SEQ ID NO: 6 - DNA, 27 bp, artificial sequence
  Synthetic
tgtatagatgagcaggtcaaatttatg

SEQ ID NO: 7 - DNA, 25 bp, artificial sequence
  Synthetic
ttagatcctgaggaaaaccatacag

SEQ ID NO: 8 - DNA, 25 bp, artificial sequence
  Synthetic
tgtgtatcactttgcttctttgagt

SEQ ID NO: 9 - DNA, 20 bp, artificial sequence
  Synthetic
ccttgggacctgagtagacg

SEQ ID NO: 10 - DNA, 20 bp, artificial sequence
  Synthetic
ccttgggacctgagtagacg

SEQ ID NO: 11 - DNA, 22 bp, artificial sequence
  Synthetic
cgatactgggtagccttcaatg

SEQ ID NO: 12 - DNA, 24 bp, artificial sequence
  Synthetic
ggaggtgtacttggtagcataacc

SEQ ID NO: 13 - DNA, 26 bp, artificial sequence
  Synthetic
gtgttatacatgtcgagtttacttcc

SEQ ID NO: 14 - DNA, 25 bp, artificial sequence
  Synthetic
gtcatattgttgtcccttcttccgc

SEQ ID NO: 15 - DNA, 18 bp, artificial sequence
  Synthetic
gagaagctgccagcagga

SEQ ID NO: 16 - DNA, 18 bp, artificial sequence
  Synthetic
tcctggagctggtggttc